SEQUENCE LISTING (37 sequences)

SEQ ID NO: 1 - RNA, 36 nt, synthetic construct
  source 1..36: mol_type = other RNA
  modified_base 1: mod_base = OTHER; note = 2'-O-methyluridine with a 3'-phosphorothioate linkage
  modified_base 2: mod_base = um
  modified_base 3: mod_base = gm
  modified_base 4: mod_base = cm
  modified_base 5: mod_base = cm
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorouridine
  modified_base 12: mod_base = gm
  modified_base 13: mod_base = gm
  modified_base 14: mod_base = um
  modified_base 15: mod_base = cm
  modified_base 16: mod_base = OTHER; note = 2'-O-methyladenosine
  modified_base 17: mod_base = um
  modified_base 18: mod_base = cm
  modified_base 19: mod_base = um
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25: mod_base = cm
  modified_base 26: mod_base = cm
  modified_base 27: mod_base = gm
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm
ttgccaagcttggtcatctagcagccgaaaggctgc

SEQ ID NO: 2 - RNA, 22 nt, synthetic construct
  source 1..22: mol_type = other RNA
  modified_base 1: mod_base = OTHER; note = MePhosphonate-4O-2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-fluoroguanosine with a 3'-phosphorothioate linkage
  modified_base 4: mod_base = OTHER; note = 2'-fluoroadenosine
  modified_base 5: mod_base = OTHER; note = 2'-fluorouridine
  modified_base 6: mod_base = gm
  modified_base 7: mod_base = OTHER; note = 2'-fluoroadenosine
  modified_base 8: mod_base = cm
  modified_base 9: mod_base = cm
  modified_base 10: mod_base = OTHER; note = 2'-fluoroadenosine
  modified_base 11: mod_base = OTHER; note = 2'-O-methyladenosine
  modified_base 12: mod_base = gm
  modified_base 13: mod_base = cm
  modified_base 14: mod_base = OTHER; note = 2'-fluorouridine
  modified_base 15: mod_base = um
  modified_base 16: mod_base = gm
  modified_base 17: mod_base = gm
  modified_base 18: mod_base = cm
  modified_base 19: mod_base = OTHER; note = 2'-O-methyladenosine
  modified_base 20: mod_base = OTHER; note = 2'-O-methyladenosine with a 3'-phosphorothioate linkage
  modified_base 21: mod_base = OTHER; note = 2'-O-methylguanosine with a 3'-phosphorothioate linkage
  modified_base 22: mod_base = gm
tagatgaccaagcttggcaagg

SEQ ID NO: 3 - RNA, 36 nt, synthetic construct
  source 1..36: mol_type = other RNA
  modified_base 1: mod_base = OTHER; note = 2'-O-methyluridine with a 3'-phosphorothioate linkage
  modified_base 2: mod_base = cm
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = gm
  modified_base 13: mod_base = gm
  modified_base 14: mod_base = um
  modified_base 15: mod_base = um
  modified_base 16: mod_base = OTHER; note = 2'-O-methyladenosine
  modified_base 17: mod_base = um
  modified_base 18: mod_base = OTHER; note = 2'-O-methyladenosine
  modified_base 19: mod_base = cm
  modified_base 20: mod_base = OTHER; note = 2'-O-methyladenosine
  modified_base 21: mod_base = gm
  modified_base 22: mod_base = cm
  modified_base 23: mod_base = OTHER; note = 2'-O-methyladenosine
  modified_base 24: mod_base = gm
  modified_base 25: mod_base = cm
  modified_base 26: mod_base = cm
  modified_base 27: mod_base = gm
  modified_base 28: mod_base = OTHER; note = 2'-aminodiethoxymethanol-Adenine-GalNAc
  modified_base 29: mod_base = OTHER; note = 2'-aminodiethoxymethanol-Adenine-GalNAc
  modified_base 30: mod_base = OTHER; note = 2'-aminodiethoxymethanol-Adenine-GalNAc
  modified_base 31: mod_base = gm
  modified_base 32: mod_base = gm
  modified_base 33: mod_base = cm
  modified_base 34: mod_base = um
  modified_base 35: mod_base = gm
  modified_base 36: mod_base = cm
tcaaaatggaaggttatacagcagccgaaaggctgc

SEQ ID NO: 4 - RNA, 22 nt, synthetic construct
  source 1..22: mol_type = other RNA
  modified_base 1: mod_base = OTHER; note = MePhosphonate-4O-2'-O-methyluridine with a 3'-phosphorothioate linkage
  modified_base 2: mod_base = OTHER; note = 2'-fluoroguanosine with a 3'-phosphorothioate linkage
  modified_base 3: mod_base = OTHER; note = 2'-fluorouridine with a 3'-phosphorothioate linkage
  modified_base 4: mod_base = OTHER; note = 2'-fluoroadenosine
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = cm
  modified_base 10: mod_base = OTHER; note = 2'-fluorouridine
  modified_base 11: mod_base = um
  modified_base 12: mod_base = cm
  modified_base 13: mod_base = cm
  modified_base 14: mod_base = OTHER; note = 2'-fluoroadenosine
  modified_base 15: mod_base = um
  modified_base 16: mod_base = um
  modified_base 17: mod_base = um
  modified_base 18: mod_base = um
  modified_base 19: mod_base = gm
  modified_base 20: mod_base = OTHER; note = 2'-O-methyladenosine with a 3'-phosphorothioate linkage
  modified_base 21: mod_base = OTHER; note = 2'-O-methylguanosine with a 3'-phosphorothioate linkage
  modified_base 22: mod_base = gm
tgtataaccttccattttgagg

SEQ ID NO: 5 - RNA, 14 nt, synthetic construct
  source 1..14: mol_type = other RNA
  modified_base 1: mod_base = OTHER; note = 2'-O-methyluridine with a 3'-phosphorothioate linkage
  modified_base 2: mod_base = um
  modified_base 3: mod_base = gm
  modified_base 4: mod_base = cm
  modified_base 5: mod_base = cm
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-fluorouridine
  modified_base 11: mod_base = OTHER; note = 2'-fluorouridine
  modified_base 12: mod_base = gm
  modified_base 13: mod_base = gm
  modified_base 14: mod_base = um
ttgccaagcttggt

SEQ ID NO: 6 - RNA, 10 nt, synthetic construct
  source 1..10: mol_type = other RNA
  modified_base 1: mod_base = OTHER; note = 2'-O-methylcytidine with a 5' phosphate cap
  modified_base 2: mod_base = OTHER; note = 2'-O-methyladenosine
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = gm
catctagcag

SEQ ID NO: 7 - RNA, 12 nt, synthetic construct
  source 1..12: mol_type = other RNA
  modified_base 1: mod_base = OTHER; note = 2'-O-methylcytidine with a 5' phosphate cap
  modified_base 2: mod_base = cm
  modified_base 3: mod_base = gm
  modified_base 4: mod_base = OTHER; note = 2'-aminodiethoxymethanol-Adenine-GalNAc
  modified_base 5: mod_base = OTHER; note = 2'-aminodiethoxymethanol-Adenine-GalNAc
  modified_base 6: mod_base = OTHER; note = 2'-aminodiethoxymethanol-Adenine-GalNAc
  modified_base 7: mod_base = gm
  modified_base 8: mod_base = gm
  modified_base 9: mod_base = cm
  modified_base 10: mod_base = um
  modified_base 11: mod_base = gm
  modified_base 12: mod_base = cm
ccgaaaggctgc

SEQ ID NO: 8 - RNA, 12 nt, synthetic construct
  source 1..12: mol_type = other RNA
  modified_base 1: mod_base = OTHER; note = 2'-O-methyladenosine with a 5' phosphate cap
  modified_base 2: mod_base = gm
  modified_base 3: mod_base = cm
  modified_base 4: mod_base = OTHER; note = 2'-fluorouridine
  modified_base 5: mod_base = um
  modified_base 6: mod_base = gm
  modified_base 7: mod_base = gm
  modified_base 8: mod_base = cm
  modified_base 9: mod_base = OTHER; note = 2'-O-methyladenosine
  modified_base 10: mod_base = OTHER; note = 2'-O-methyladenosine with a 3'-phosphorothioate linkage
  modified_base 11: mod_base = OTHER; note = 2'-O-methylguanosine with a 3'-phosphorothioate linkage
  modified_base 12: mod_base = gm
agcttggcaagg

SEQ ID NO: 9 - RNA, 10 nt, synthetic construct
  source 1..10: mol_type = other RNA
  modified_base 1: mod_base = OTHER; note = MePhosphonate-4O-2'-O-methyluridine with a 3'-phosphorothioate linkage
  modified_base 2: mod_base = OTHER; note = 2'-fluoroadenosine with a 3'-phosphorothioate linkage
  modified_base 3: mod_base = OTHER; note = 2'-fluoroguanosine with a 3'-phosphorothioate linkage
  modified_base 4: mod_base = OTHER; note = 2'-fluoroadenosine
  modified_base 5: mod_base = OTHER; note = 2'-fluorouridine
  modified_base 6: mod_base = gm
  modified_base 7: mod_base = OTHER; note = 2'-fluoroadenosine
  modified_base 8: mod_base = cm
  modified_base 9: mod_base = cm
  modified_base 10: mod_base = OTHER; note = 2'-fluoroadenosine
tagatgacca

SEQ ID NO: 10 - RNA, 12 nt, synthetic construct
  source 1..12: mol_type = other RNA
  modified_base 1: mod_base = OTHER; note = 2'-O-methylcytidine with a 5' phosphate cap
  modified_base 2: mod_base = OTHER; note = 2'-O-methyladenosine
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = gm
  modified_base 11: mod_base = cm
  modified_base 12: mod_base = cm
catctagcagcc

SEQ ID NO: 11 - RNA, 10 nt, synthetic construct
  source 1..10: mol_type = other RNA
  modified_base 1: mod_base = OTHER; note = 2'-O-methylguanosine with a 5' phosphate cap
  modified_base 2: mod_base = OTHER; note = 2'-aminodiethoxymethanol-Adenine-GalNAc
  modified_base 3: mod_base = OTHER; note = 2'-aminodiethoxymethanol-Adenine-GalNAc
  modified_base 4: mod_base = OTHER; note = 2'-aminodiethoxymethanol-Adenine-GalNAc
  modified_base 5: mod_base = gm
  modified_base 6: mod_base = gm
  modified_base 7: mod_base = cm
  modified_base 8: mod_base = um
  modified_base 9: mod_base = gm
  modified_base 10: mod_base = cm
gaaaggctgc

SEQ ID NO: 12 - RNA, 11 nt, synthetic construct
  source 1..11: mol_type = other RNA
  modified_base 1: mod_base = OTHER; note = 2'-O-methylcytidine with a 5' phosphate cap
  modified_base 2: mod_base = OTHER; note = 2'-O-methyladenosine
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = gm
  modified_base 11: mod_base = cm
catctagcagc

SEQ ID NO: 13 - RNA, 11 nt, synthetic construct
  source 1..11: mol_type = other RNA
  modified_base 1: mod_base = OTHER; note = 2'-O-methylcytidine with a 5' phosphate cap
  modified_base 2: mod_base = gm
  modified_base 3: mod_base = OTHER; note = 2'-aminodiethoxymethanol-Adenine-GalNAc
  modified_base 4: mod_base = OTHER; note = 2'-aminodiethoxymethanol-Adenine-GalNAc
  modified_base 5: mod_base = OTHER; note = 2'-aminodiethoxymethanol-Adenine-GalNAc
  modified_base 6: mod_base = gm
  modified_base 7: mod_base = gm
  modified_base 8: mod_base = cm
  modified_base 9: mod_base = um
  modified_base 11: mod_base = cm
  modified_base 10: mod_base = gm
cgaaaggctgc

SEQ ID NO: 14 - RNA, 10 nt, synthetic construct
  source 1..10: mol_type = other RNA
  modified_base 1: mod_base = OTHER; note = 2'-O-methyluridine with a 3'-phosphorothioate linkage
  modified_base 2: mod_base = um
  modified_base 3: mod_base = gm
  modified_base 4: mod_base = cm
  modified_base 5: mod_base = cm
  modified_base 6: mod_base = OTHER; note = 2'-O-methyladenosine
  modified_base 7: mod_base = OTHER; note = 2'-O-methyladenosine
  modified_base 8: mod_base = OTHER; note = 2'-fluoroguanosine
  modified_base 9: mod_base = OTHER; note = 2'-fluorocytidine
  modified_base 10: mod_base = OTHER; note = 2'-fluorouridine
ttgccaagct

SEQ ID NO: 15 - RNA, 14 nt, synthetic construct
  source 1..14: mol_type = other RNA
  modified_base 1: mod_base = OTHER; note = 2'-fluorouridine with a 5' phosphate cap
  modified_base 2: mod_base = gm
  modified_base 3: mod_base = gm
  modified_base 4: mod_base = um
  modified_base 5: mod_base = cm
  modified_base 6: mod_base = OTHER; note = 2'-O-methyladenosine
  modified_base 7: mod_base = um
  modified_base 8: mod_base = cm
  modified_base 9: mod_base = um
  modified_base 10: mod_base = OTHER; note = 2'-O-methyladenosine
  modified_base 11: mod_base = gm
  modified_base 12: mod_base = cm
  modified_base 13: mod_base = OTHER; note = 2'-O-methyladenosine
  modified_base 14: mod_base = gm
tggtcatctagcag

SEQ ID NO: 16 - RNA, 16 nt, synthetic construct
  source 1..16: mol_type = other RNA
  modified_base 1: mod_base = OTHER; note = 2'-fluoroadenosine with a 5' phosphate cap
  modified_base 2: mod_base = cm
  modified_base 3: mod_base = cm
  modified_base 4: mod_base = OTHER; note = 2'-fluoroadenosine
  modified_base 5: mod_base = OTHER; note = 2'-O-methyladenosine
  modified_base 6: mod_base = gm
  modified_base 7: mod_base = cm
  modified_base 8: mod_base = OTHER; note = 2'-fluorouridine
  modified_base 9: mod_base = um
  modified_base 10: mod_base = gm
  modified_base 11: mod_base = gm
  modified_base 12: mod_base = cm
  modified_base 13: mod_base = OTHER; note = 2'-O-methyladenosine
  modified_base 14: mod_base = OTHER; note = 2'-O-methyladenosine with a 3'-phosphorothioate linkage
  modified_base 15: mod_base = OTHER; note = 2'-O-methylguanosine with a 3'-phosphorothioate linkage
  modified_base 16: mod_base = gm
accaagcttggcaagg

SEQ ID NO: 17-18 - skipped (2 entries)

SEQ ID NO: 19 - RNA, 15 nt, synthetic construct
  source 1..15: mol_type = other RNA
  modified_base 1: mod_base = OTHER; note = 2'-fluorouridine with a 5' phosphate cap
  modified_base 2: mod_base = OTHER; note = 2'-fluorouridine
  modified_base 3: mod_base = gm
  modified_base 4: mod_base = gm
  modified_base 5: mod_base = um
  modified_base 6: mod_base = cm
  modified_base 7: mod_base = OTHER; note = 2'-O-methyladenosine
  modified_base 8: mod_base = um
  modified_base 9: mod_base = cm
  modified_base 10: mod_base = um
  modified_base 11: mod_base = OTHER; note = 2'-O-methyladenosine
  modified_base 12: mod_base = gm
  modified_base 13: mod_base = cm
  modified_base 14: mod_base = OTHER; note = 2'-O-methyladenosine
  modified_base 15: mod_base = gm
ttggtcatctagcag

SEQ ID NO: 20 - RNA, 15 nt, synthetic construct
  source 1..15: mol_type = other RNA
  modified_base 1: mod_base = OTHER; note = 2'-O-methylcytidine with a 5' phosphate cap
  modified_base 2: mod_base = cm
  modified_base 3: mod_base = OTHER; note = 2'-fluoroadenosine
  modified_base 4: mod_base = OTHER; note = 2'-O-methyladenosine
  modified_base 5: mod_base = gm
  modified_base 6: mod_base = cm
  modified_base 7: mod_base = OTHER; note = 2'-fluorouridine
  modified_base 8: mod_base = um
  modified_base 9: mod_base = gm
  modified_base 10: mod_base = gm
  modified_base 11: mod_base = cm
  modified_base 12: mod_base = OTHER; note = 2'-O-methyladenosine
  modified_base 13: mod_base = OTHER; note = 2'-O-methyladenosine with a 3'-phosphorothioate linkage
  modified_base 14: mod_base = OTHER; note = 2'-O-methylguanosine with a 3'-phosphorothioate linkage
  modified_base 15: mod_base = gm
ccaagcttggcaagg

SEQ ID NO: 21-22 - skipped (2 entries)

SEQ ID NO: 23 - RNA, 16 nt, synthetic construct
  source 1..16: mol_type = other RNA
  modified_base 1: mod_base = OTHER; note = 2'-fluorocytidine with a 5' phosphate cap
  modified_base 2: mod_base = OTHER; note = 2'-fluorouridine
  modified_base 3: mod_base = OTHER; note = 2'-fluorouridine
  modified_base 4: mod_base = gm
  modified_base 5: mod_base = gm
  modified_base 6: mod_base = um
  modified_base 7: mod_base = cm
  modified_base 8: mod_base = OTHER; note = 2'-O-methyladenosine
  modified_base 9: mod_base = um
  modified_base 10: mod_base = cm
  modified_base 11: mod_base = um
  modified_base 12: mod_base = OTHER; note = 2'-O-methyladenosine
  modified_base 13: mod_base = gm
  modified_base 14: mod_base = cm
  modified_base 15: mod_base = OTHER; note = 2'-O-methyladenosine
  modified_base 16: mod_base = gm
cttggtcatctagcag

SEQ ID NO: 24 - RNA, 14 nt, synthetic construct
  source 1..14: mol_type = other RNA
  modified_base 1: mod_base = OTHER; note = 2'-O-methylcytidine with a 5' phosphate cap
  modified_base 2: mod_base = OTHER; note = 2'-fluoroadenosine
  modified_base 3: mod_base = OTHER; note = 2'-O-methyladenosine
  modified_base 4: mod_base = gm
  modified_base 5: mod_base = cm
  modified_base 6: mod_base = OTHER; note = 2'-fluorouridine
  modified_base 7: mod_base = um
  modified_base 8: mod_base = gm
  modified_base 9: mod_base = gm
  modified_base 10: mod_base = cm
  modified_base 11: mod_base = OTHER; note = 2'-O-methyladenosine
  modified_base 12: mod_base = OTHER; note = 2'-O-methyladenosine with a 3'-phosphorothioate linkage
  modified_base 13: mod_base = OTHER; note = 2'-O-methylguanosine with a 3'-phosphorothioate linkage
  modified_base 14: mod_base = gm
caagcttggcaagg

SEQ ID NO: 25 - skipped

SEQ ID NO: 26 - RNA, 13 nt, synthetic construct
  source 1..13: mol_type = other RNA
  modified_base 1: mod_base = OTHER; note = 2'-O-methylcytidine with a 5' phosphate cap
  modified_base 2: mod_base = OTHER; note = 2'-O-methyladenosine
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = gm
  modified_base 11: mod_base = cm
  modified_base 12: mod_base = cm
  modified_base 13: mod_base = gm
catctagcagccg

SEQ ID NO: 27 - skipped

SEQ ID NO: 28 - RNA, 18 nt, synthetic construct
  source 1..18: mol_type = other RNA
  modified_base 1: mod_base = OTHER; note = 2'-O-methylcytidine with a 5' phosphate cap
  modified_base 2: mod_base = OTHER; note = 2'-O-methyladenosine
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = gm
  modified_base 11: mod_base = cm
  modified_base 12: mod_base = cm
  modified_base 13: mod_base = gm
  modified_base 14: mod_base = OTHER; note = 2'-aminodiethoxymethanol-Adenine-GalNAc
  modified_base 15: mod_base = OTHER; note = 2'-aminodiethoxymethanol-Adenine-GalNAc
  modified_base 16: mod_base = OTHER; note = 2'-aminodiethoxymethanol-Adenine-GalNAc
  modified_base 17: mod_base = gm
  modified_base 18: mod_base = gm
catctagcagccgaaagg

SEQ ID NO: 29-31 - skipped (3 entries)

SEQ ID NO: 32 - RNA, 14 nt, synthetic construct
  source 1..14: mol_type = other RNA
  modified_base 1: mod_base = OTHER; note = 2'-O-methyluridine with a 3'-phosphorothioate linkage
  modified_base 2: mod_base = cm
  modified_base 3: mod_base = OTHER; note = 2'-O-methyladenosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = um
  modified_base 8: mod_base = OTHER; note = 2'-fluoroguanosine
  modified_base 9: mod_base = OTHER; note = 2'-fluoroguanosine
  modified_base 10: mod_base = OTHER; note = 2'-fluoroadenosine
  modified_base 11: mod_base = OTHER; note = 2'-fluoroadenosine
  modified_base 12: mod_base = gm
  modified_base 13: mod_base = gm
  modified_base 14: mod_base = um
tcaaaatggaaggt

SEQ ID NO: 33 - RNA, 10 nt, synthetic construct
  source 1..10: mol_type = other RNA
  modified_base 1: mod_base = OTHER; note = 2'-O-methyluridine with a 5' phosphate cap
  modified_base 2: mod_base = OTHER; note = 2'-O-methyladenosine
  modified_base 3: mod_base = um
  modified_base 4: mod_base = OTHER; note = 2'-O-methyladenosine
  modified_base 5: mod_base = cm
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = gm
tatacagcag

SEQ ID NO: 34 - RNA, 12 nt, synthetic construct
  source 1..12: mol_type = other RNA
  modified_base 1: mod_base = OTHER; note = 2'-O-methyluridine with a 5' phosphate cap
  modified_base 2: mod_base = cm
  modified_base 3: mod_base = cm
  modified_base 4: mod_base = OTHER; note = 2'-fluoroadenosine
  modified_base 5: mod_base = um
  modified_base 6: mod_base = um
  modified_base 7: mod_base = um
  modified_base 8: mod_base = um
  modified_base 9: mod_base = gm
  modified_base 10: mod_base = OTHER; note = 2'-O-methyladenosine with a 3'-phosphorothioate linkage
  modified_base 11: mod_base = OTHER; note = 2'-O-methylguanosine with a 3'-phosphorothioate linkage
  modified_base 12: mod_base = gm
tccattttgagg

SEQ ID NO: 35 - RNA, 10 nt, synthetic construct
  source 1..10: mol_type = other RNA
  modified_base 1: mod_base = OTHER; note = MePhosphonate-4O-2'-O-methyluridine with a 3'-phosphorothioate linkage
  modified_base 2: mod_base = OTHER; note = 2'-fluoroguanosine with a 3'-phosphorothioate linkage
  modified_base 3: mod_base = OTHER; note = 2'-fluorouridine with a 3'-phosphorothioate linkage
  modified_base 4: mod_base = OTHER; note = 2'-fluoroadenosine
  modified_base 5: mod_base = OTHER; note = 2'-fluorouridine
  modified_base 6: mod_base = OTHER; note = 2'-O-methyladenosine
  modified_base 7: mod_base = OTHER; note = 2'-fluoroadenosine
  modified_base 8: mod_base = cm
  modified_base 9: mod_base = cm
  modified_base 10: mod_base = OTHER; note = 2'-fluorouridine
tgtataacct

SEQ ID NO: 36 - RNA, 22 nt, synthetic construct
  source 1..22: mol_type = other RNA
  modified_base 1: mod_base = OTHER; note = 2'-O-methylcytidine with a 5' phosphate cap
  modified_base 2: mod_base = OTHER; note = 2'-O-methyladenosine
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = gm
  modified_base 11: mod_base = cm
  modified_base 12: mod_base = cm
  modified_base 13: mod_base = gm
  modified_base 14: mod_base = OTHER; note = 2'-aminodiethoxymethanol-Adenine-GalNAc
  modified_base 15: mod_base = OTHER; note = 2'-aminodiethoxymethanol-Adenine-GalNAc
  modified_base 16: mod_base = OTHER; note = 2'-aminodiethoxymethanol-Adenine-GalNAc
  modified_base 17: mod_base = gm
  modified_base 18: mod_base = gm
  modified_base 19: mod_base = cm
  modified_base 20: mod_base = um
  modified_base 21: mod_base = gm
  modified_base 22: mod_base = cm
catctagcagccgaaaggctgc

SEQ ID NO: 37 - RNA, 22 nt, synthetic construct
  source 1..22: mol_type = other RNA
  modified_base 1: mod_base = OTHER; note = 2'-O-methylcytidine where the 5' end of the sequence is linked to the 3' end at 2'-O-methyluridine
  modified_base 2: mod_base = OTHER; note = 2'-O-methyladenosine
  modified_base 3: mod_base = um
  modified_base 4: mod_base = cm
  modified_base 5: mod_base = um
  modified_base 6: mod_base = OTHER; note = 2'-O-methyladenosine
  modified_base 7: mod_base = gm
  modified_base 8: mod_base = cm
  modified_base 9: mod_base = OTHER; note = 2'-O-methyladenosine
  modified_base 10: mod_base = gm
  modified_base 11: mod_base = cm
  modified_base 12: mod_base = cm
  modified_base 13: mod_base = gm
  modified_base 14: mod_base = OTHER; note = 2'-aminodiethoxymethanol-Adenine-GalNAc
  modified_base 15: mod_base = OTHER; note = 2'-aminodiethoxymethanol-Adenine-GalNAc
  modified_base 16: mod_base = OTHER; note = 2'-aminodiethoxymethanol-Adenine-GalNAc
  modified_base 17: mod_base = gm
  modified_base 18: mod_base = gm
  modified_base 19: mod_base = cm
  modified_base 20: mod_base = um
  modified_base 21: mod_base = gm
  modified_base 22: mod_base = cm
ctagcagccgaaaggctgccat

(19) United States
(12) Patent Application Publication
(10) Pub. No.: US 2026/0226459 A1
(43) Pub. Date: Aug. 6, 2026

(54) OLIGONUCLEOTIDE FRAGMENTS AND METHODS OF MAKING RNAI AGENTS USING THE SAME

(71) Applicant: ELI LILLY AND COMPANY, Indianapolis, IN (US)

(72) Inventors: Caleb Christopher CULY, Greenfield, IN (US); Scott MAY, Fishers, IN (US); Eric MOHER, Greenwood, IN (US); Sergey TSUKANOV, Carmel, IN (US)

(21) Appl. No.: 19/141,381

(22) PCT Filed: Dec. 19, 2023
(86) PCT No.: PCT/US2023/084807

§ 371 Date: Jun. 20, 2025

Related U.S. Application Data
(60) Provisional application No. 63/434,661, filed on Dec. 22, 2022.

(51) Int. Cl. C12N 15/113; C12N 9/00; C12P 19/34

(52) U.S. Cl. CPC C12N 15/113; C12N 9/93; C12P 19/34; C12Y 605/01003; C12N 2310/14; C12N 2330/50

(57) ABSTRACT

Intermediate compounds (i.e., oligonucleotide fragments) are disclosed for making RNAi agents, or pharmaceutically acceptable salts thereof. In addition, methods are disclosed for making single-stranded oligonucleotides by ligating two or more of the intermediate compounds herein via a hybrid chemical-enzymatic route.

### REFERENCE TO RELATED APPLICATIONS

This application claims the full Paris Convention benefit of, and priority to, United States provisional patent application numbered 63/434,661, filed on Dec. 22, 2022.

### REFERENCE TO SEQUENCE LISTING SUBMITTED ELECTRONICALLY

The disclosure is being filed along with a Sequence Listing in ST.26 XML format. The Sequence Listing is provided as a file titled “30019_US_PRI” created 24 Oct. 2023 and is 35 kilobytes (kb) in size. The Sequence Listing information in the ST.26 XML format is incorporated herein by reference in its entirety.

### TECHNICAL FIELD

The disclosure relates generally to biology, chemistry and medicine, and more particularly it relates to methods of synthesizing oligonucleotides via a hybrid chemical-enzymatic route.

### BACKGROUND

Oligonucleotides are used in various biological and biochemical applications. Of interest herein, is the use of oligonucleotide as therapeutic agents such RNA activation (RNAa), RNA editing (RNAe) and RNA interference (RNAi). Such widespread use of oligonucleotides results in an increasing demand for rapid, inexpensive and efficient methods for their synthesis.

Oligonucleotides can be synthesized via a number of methods known in the art, especially via solid-phase synthesis by repeated coupling of nucleoside phosphoramidites. See, e.g., Beaucage & Caruthers (1981) Tetrahedron Letters 22:1859-1862; McBride & Caruthers (1983) Tetrahedron Letters 24:245-248; Sinha et al. (1984) Nucleic Acids Res. 12:4539-4557 and Beaucage & Iyer (1992) Tetrahedron 48:2223-2311.

There is a need, however, for alternative methods of making oligonucleotides and intermediates thereof to enable pharmaceutically elegant production with commercially desired purity. Likewise, there is a need for efficient methods and stable intermediates to provide oligonucleotides efficiently, with fewer purification steps.

### BRIEF SUMMARY

To address this need, the disclosure describes nucleotide-based intermediate compounds (i.e., oligonucleotide fragments), as well as methods of making single-stranded (ss) oligonucleotides by ligating a plurality of the oligonucleotide fragments herein and ultimately making double-stranded (ds) therapeutic oligonucleotides such as RNAi agents.

With regard to the intermediate compounds, the disclosure describes oligonucleotide fragments having a nucleotide sequence selected from any one of SEQ ID NOS:5 to 37.

With regard to the methods, the disclosure describes methods of making a nucleic acid having a nucleotide sequence of SEQ ID NO:1 that includes a step of ligating the following combination of oligonucleotide fragments having nucleotide sequences selected from:
(a) SEQ ID NOS:5, 6 and 7;
(b) SEQ ID NOS:5, 10 and 11;
(c) SEQ ID NOS:5, 12 and 13;
(d) SEQ ID NOS:7, 14 and 15;
(e) SEQ ID NOS:7, 18 and 19;
(f) SEQ ID NOS: 7, 22 and 23; and
(g) SEQ ID NOS: 5, 26 and 27.

In addition, the disclosure describes methods of making a nucleic acid having a nucleotide sequence of SEQ ID NO:2 that includes a step of ligating the following combinations of oligonucleotide fragments having nucleotide sequences selected from:
(a′) SEQ ID NOS:8 and 9;
(b′) SEQ ID NOS:16 and 17;
(c′) SEQ ID NOS:20 and 21; and
(d′) SEQ ID NOS:24 and 25.

The methods above can include an additional step of annealing SEQ ID NO:1 and SEQ ID NO:2 to form a RNAi agent that modulates apolipoprotein(a) gene (LPA) expression.

Moreover, the disclosure describes methods of making a nucleic acid having a nucleotide sequence of SEQ ID NO:3 that includes a step of ligating the following combination of oligonucleotide fragments having nucleotide sequences selected from:
(a) SEQ ID NOS:7, 32 and 33.

In addition, the disclosure describes methods of making a nucleic acid having a nucleotide sequence of SEQ ID NO:4 that includes a step of ligating the following combination of oligonucleotide fragments having nucleotide sequences selected from:
(a′) SEQ ID NOS:34 and 35.

The methods above can include an additional step of annealing SEQ ID NO:3 and SEQ ID NO:4 to form a RNAi agent that modulates angiopoietin-like 3 gene (ANGPTL3) expression.

In any of the above methods, the ligating can be mediated by an enzyme. In some instances, the enzyme is a ligase such as a naturally occurring ligase or a non-naturally occurring ligase. In other instances, the ligase is a DNA ligase. In certain instances, the ligase is a RNA ligase.

An advantage of the methods herein includes process improvements such as, for example, shorter fragments initially produced via solid phase oligonucleotide synthesis (SPOS) allow for increased purity and higher yields. With shorter fragments, more route flexibility is available to incorporate modified nucleotides, and an ability to redesign fragment structures to address more difficult segments of the strands.

An advantage of the methods herein includes an improved control strategy for impurities during the synthesis, which can include improved detection and characterization of impurities at a fragment stage and an improved final impurity profile for a crude oligonucleotide duplex.

An advantage of the methods herein includes improved final duplex purity due to rejection of certain classes of fragment impurities based on their inability to participate in the ligation step or reduced adherence to the complementarity principle during fragment self-assembly in the annealing step.

An advantage of the methods herein includes reduced in unit operations required to produce an oligonucleotide duplex as a single step used to form a duplex material from fragment building blocks allowing to reduce downstream operations to a single chromatography and ultrafiltration (vs need for a separate step for each strand as in a conventional approach).

An advantage of the methods herein includes that synthesis of shorter fragments via SPOS can allow for reduced washing cycles and for reduced volumes of reagents, leading to a reduced process mass intensity (PMI).

An advantage of the methods herein includes that with shorter fragment intermediates, the implications of synthesis failure during fragment manufacturing are diminished, resulting in reduced total cost and manufacturing cycle impact.

An advantage of the methods herein includes that shorter fragment intermediates are more amenable to new synthetic manufacturing platforms, and enzymatic assembly of an oligonucleotide duplex allows for introducing other innovative technologies for downstream unit operations.

An advantage of the methods herein includes flexibility in supply chain and logistics of the manufacturing process by using several independent fragments.

An advantage of the methods herein includes further flexibility in supply chain resulting from sequences utilizing the same delivery platform to enable designing conservative fragments with identical nucleotide sequence composition.

An advantage of the methods herein includes that use of parallel manufacturing of fragments can reduce manufacturing cycles by parallel processing of the fragments.

An advantage of the methods herein includes that current good manufacturing practice (cGMP) enzymatic ligation step can be executed under water-based conditions at a solvent-free facility without a need for specialized equipment.

### BRIEF DESCRIPTION OF THE DRAWINGS

The advantages, effects, features, and objects other than those set forth above will become more readily apparent when consideration is given to the detailed description below. Such detailed description refers to the following drawing(s), where:

FIG. 1 shows a structural representation of an exemplary RNAi agent (SEQ ID NO:1 and 2) that has a nicked tetraloop structure and that modulates LPA expression.

FIG. 2 shows a structural representation of an exemplary RNAi agent (SEQ ID NOS:3 and 4) that has a nicked tetraloop structure and that modulates ANGTPL3 expression.

### DETAILED DESCRIPTION

### Overview

Intl. Patent Application Publication No. WO 2022/032288 describes a RNAi agent (e.g., LPA-3291-Ml) that can be used for attenuating, preventing and/or treating diseases, disorders and/or conditions associated with LPA expression (i.e., reduce the levels of LPA mRNA and Apo(a) protein, as well as reduce Lp(a) level). The RNAi agent includes N-acetylgalactosamine (GalNAc) ligands to target it to the asialoglycoprotein receptor (ASGPR).

Intl. Patent Application Publication No. WO 2021/188795 describes a RNAi agent (e.g., GalXC-1412) that can be used for attenuating, preventing and/or treating diseases, disorders and/or conditions associated with ANGPTL3 expression (i.e., reduce the levels of ANGPTL3 mRNA and ANGPTL3 protein). The RNAi agent includes GalNAc ligands to target it to the ASGPR.

### Abbreviations and Definitions

Unless defined otherwise, all technical and scientific terms used herein have the same meaning as commonly understood by one of skill in the art to which the disclosure pertains. Although any methods and materials similar to or equivalent to those described herein can be used in the practice or testing of the incretin analog, pharmaceutical compositions and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

Certain abbreviations used herein are defined as follows:

“A” refers to adenosine; “G” refers to guanosine; “U” refers to uridine; “C” refers to cytosine; “fX” refers to a 2′-fluoro-nucleotide (e.g., fA, fG, fU, fC); “fX^{s}” refers to 2′-fluoro nucleotide attached via phosphorothioate linkage; “mX” refers to a 2′-O-methyl nucleotide (e.g., mA, mG, mU, mC); “mX^{s}” refers to a 2′-O-methyl nucleotide attached via phosphorothioate linkage (e.g., mA^{S}, mA^{S}, mU^{S}, mC^{S}); “adem A” refers to 2′-O-GalNAc-modified adenosine (sodium salt equivalent), which has the following structure:

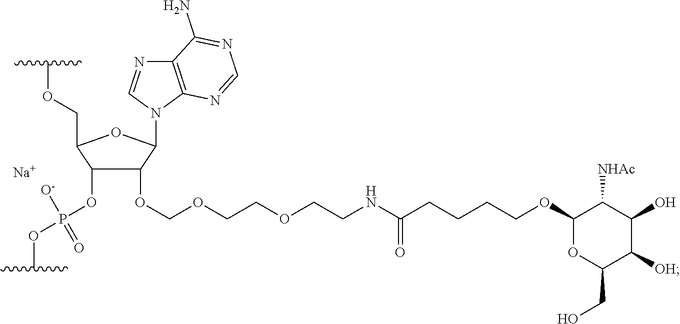 [SMILES: [Na+] ; *OCC1OC(n2cnc3c(N)ncnc32)C(OCOCCOCCNC(=O)CCCCO[C@@H]2O[C@H](CO)[C@H](O)[C@H](O)[C@H]2NC(C)=O)C1OP(=O)([O-])O*]

“ACN” refers to acetonitrile (C₂H₃N); “ANGPTL3” refers to angiopoietin-like 3 gene; “ASGPR” refers to asialoglycoprotein receptor; “cGMP” refers to current good manufacturing practice; “CV” refers to column volume(s); “Da” refers to dalton(s); “DCA” refers to dichloroacetic acid (C₂H₂C₁₂O₂); “DEA” refers to diethylamine (C₄H₁₁N); “DIPEA” refers to N,N-diisopropylethylamine (C₈H₁₉N); “Dmt” refers to 4,4′-dimethoxytrityl; “DNA” refers to deoxyribonucleic acid; “ds” refers to double-stranded; “DTT” refers to 1,4-dithiothreitol, “EDTA” refers to ethylenediaminetetraacetic acid (C₁₀H₁₆N₂O₈); “EtOH” refers to ethanol (C₂H₆O); “ETT” refers to 5-(ethylthio)-1H-tetrazole (C₃H₆N₄S); “GalNAc” refers to N-acetylgalactosamine, which has the following structure:

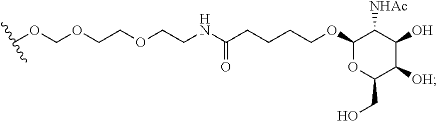 [SMILES: *OCOCCOCCNC(=O)CCCCO[C@@H]1O[C@H](CO)[C@H](O)[C@H](O)[C@H]1NC(C)=O]

“HFIP” refers to hexafluoroisopropanol (C₃H₂F₆O); “HPLC” refers to high-performance liquid chromatography; “LPA” refers to apolipoprotein(a) gene; “meMOP mU” refers to 4′-O-monomethylphosphonate-2′-O-methyl uridine (sodium salt equivalent), which has the following structure:

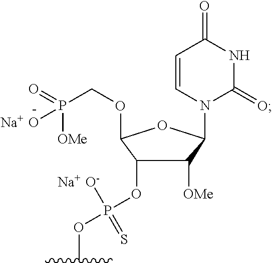

“MWCO” refers to molecular weight cutoff, “NAD” refers to nicotinamide adenine dinucleotide; “p” refers to 5′ phosphate cap; “PA” refers to phosphoramidite; “PO” refers to phosphodiester; “PS” refers to phosphorothioate; “PMI” refers to process mass intensity; “RISC” refers to RNA-induced silencing complex; “RNA” refers to ribonucleic acid; “RNAa” refers to RNA activation; “RNAe” refers to RNA editing; “RNAi” refers to RNA interference; “SPOS” refers to solid phase oligonucleotide synthesis; “ss” refers to single-stranded; “TFF” refers to tangential flow filtration; “Tₘ” refers to melting temperature; “UPLC” refers to ultra-performance liquid chromatography; “UF/DF” refers to ultrafiltration and diafiltration; and “UV” refers to ultraviolet.

Certain definitions used herein are defined as follows:

As used herein, “about” means within a statistically meaningful range of a value or values such as, for example, a stated concentration, length, molecular weight, pH, pressure, sequence identity, time frame, temperature or volume. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, “anneal,” “annealing” and the like mean hybridizing complementary oligonucleotides in a sequence-specific manner. Conditions for annealing will depend on the melting temperature (Tₘ) of the hybridized complementary oligonucleotides and will be readily apparent to one of skill in the art. For example, the annealing temperature may be below the Tₘ of the hybridized oligonucleotides. Alternatively, the annealing temperature may be close to the Tₘ of the hybridized oligonucleotides (e.g., +/−about 1° C., 2° C. or 3° C.). The annealing temperature typically is not higher than about 10° C. above the Tₘ of the hybridized oligonucleotides.

As used herein, “antisense strand” or “guide strand” means a ss oligonucleotide that is complementary to a region of a target sequence, such as a target sequence in a mRNA. Likewise, and as used herein, “sense strand” or “passenger strand” means a ss oligonucleotide that is complementary to a region of an antisense strand.

As used herein, “asialoglycoprotein receptor” or “ASGPR” means a bipartite C-type lectin formed by a major 48 kDa subunit (ASGPR-1) and minor 40 kDa subunit (ASGPR-2).

As used herein, a “chemically synthesized” oligonucleotide refers to an oligonucleotide produced by using chemical reactions, for example, without using enzymes. Methods of chemically synthesizing oligonucleotide such as RNA molecules are known in the art, in particular, the chemical synthesis methods as described in Verma & Eckstein (1998) or as described herein. Generally, ds RNA constructs can by synthesized using SPOS (see, e.g., Usman et al. (1987) J. Am. Chem. Soc. 109:7845-7854, U.S. Pat. Nos. 5,804,683; 5,831,071; 5,998,203; 6,008,400; 6,111,086; 6,117,657; 6,353,098; 6,362,323; 6,437,117 and 6,469,158; and Scaringe et al. (1990) Nucleic Acids Res. 18:5433-5441; see also, Beaucage & Caruthers (1981) Tetrahedron Letters 22:1859-1862; McBride & Caruthers (1983) Tetrahedron Letters 24:245-248; Sinha et al. (1984) Nucleic Acids Res. 12:4539-4557 and Beaucage & Iyer (1992) Tetrahedron 48:2223-2311); and Intl. Patent Application Publication Nos. 2005/070859 and 2012/157723.

As used herein, “complementary” means a structural relationship between two nucleotides (e.g., on two opposing nucleic acids or on opposing regions of a ss nucleic acid e.g., a hairpin) that permits the two nucleotides to form base pairs with one another. For example, a purine nucleotide of one oligonucleotide that is complementary to a pyrimidine nucleotide of an opposing oligonucleotide may base pair together by forming hydrogen bonds with one another. Complementary nucleotides can base pair in the Watson-Crick manner or in any other manner that allows for the formation of stable duplexes. Likewise, two oligonucleotides may have regions of multiple nucleotides that are complementary with each other to form regions of complementarity, as described herein.

As used herein, “deoxyribonucleotide” means a nucleotide having a hydrogen in place of a hydroxyl at the 2′ position of its pentose sugar when compared with a ribonucleotide. A modified deoxyribonucleotide has one or more modifications or substitutions of atoms other than hydroxyl at the 2′ position, including modifications or substitutions in or of the nucleobase, sugar, or phosphate group.

As used herein, “double-stranded oligonucleotide” or “ds oligonucleotide” means an oligonucleotide that is in a duplex form. The complementary base-pairing of duplex region(s) of a ds oligonucleotide can be formed between antiparallel sequences of nucleotides of covalently separate nucleic acids. Likewise, complementary base-pairing of duplex region(s) of a ds oligonucleotide can be formed between antiparallel sequences of nucleotides of nucleic acids that are covalently linked. Moreover, complementary base-pairing of duplex region(s) of a ds oligonucleotide can be formed from a ss nucleic acid that is folded (e.g., via a hairpin) to provide complementary antiparallel sequences of nucleotides that base pair together. A ds oligonucleotide can include two covalently separate nucleic acids that are fully duplexed with one another. However, a ds oligonucleotide can include two covalently separate nucleic acids that are partially duplexed (e.g., having overhangs at one or both ends). A ds oligonucleotide can include an antiparallel sequence of nucleotides that are partially complementary, and thus, may have one or more mismatches, which may include internal mismatches or end mismatches.

As used herein, “duplex” and “duplex region,” in reference to a nucleic acid (e.g., an oligonucleotide), means a structure formed through complementary base pairing of two antiparallel sequences of nucleotides, whether formed by two covalently separate nucleic acids or by a single, folded strand (e.g., via a hairpin).

As used herein, “enzymatic ligation,” “enzymatically ligating” and the like mean that a link between two adjacent nucleotides is formed enzymatically, where such linkage may be a naturally occurring phosphodiester (PO) bond or a modified linkage including, but not limited to, phosphorothioate (PS) bond or phosphoramidite (PA) bond.

As used herein, an “enzymatically synthesized” oligonucleotide means an oligonucleotide with modifications produced by the reaction of a nucleic acid with an enzyme, including naturally occurring enzymes or non-naturally occurring enzymes (e.g., kinases, ligases, methyltransferases, nicking enzymes, nucleases, phosphatases, sulfurylases and recombinases). Correspondingly, and as used herein, “enzymatic” modifications refer to those modifications that are produced by the reaction of a nucleic acid with an enzyme, including naturally occurring and non-naturally occurring enzymes.

As used herein, “ligase” means an enzyme that catalyzes a joining (i.e., covalent joining) of two oligonucleotides, for example, by forming a PO bond between the 3′ end of one oligonucleotide (or fragment) and the 5′ end of the same or another oligonucleotide (or fragment). These enzymes are often referred to as DNA ligases or RNA ligases and usually are members of the Enzyme Class EC 6.5 as defined by the International Union of Biochemistry and Molecular Biology (i.e., ligases used to form phosphoric ester bonds). Moreover, a ligase herein can be capable of joining an unmodified oligonucleotide to another unmodified oligonucleotide, can be capable of joining an unmodified oligonucleotide to a modified oligonucleotide (i.e., a modified 5′ oligonucleotide to an unmodified 3′ oligonucleotide and/or an unmodified 5′ oligonucleotide to a modified 3′ oligonucleotide), and/or can be capable of joining a modified oligonucleotide to another modified oligonucleotide.

As used herein, “modified ligase” or “non-naturally occurring ligase” means a ligase that differs from a naturally occurring (i.e., wild-type) ligase by one or more amino acid residues.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide, and thymidine deoxyribonucleotide. A modified nucleotide can be a non-naturally occurring nucleotide. A modified nucleotide can have, for example, one or more chemical modification in its sugar, nucleobase, and/or phosphate group. Additionally, or alternatively, a modified nucleotide can have one or more chemical moieties conjugated to a corresponding reference nucleotide.

As used herein, “N-acetylgalactosamine” or “GalNAc” means 2-(acetylamino)-2-deoxy-D-galactose or derivatives thereof, which can be directly or indirectly conjugated to an oligonucleotide herein to target the oligonucleotide to the ASGPR.

As used herein, “nicked tetraloop structure” means a structure of a RNAi agent that is characterized by separate sense and antisense strands, in which the sense strand has a region of complementarity with the antisense strand, and in which at least one of the strands, generally the sense strand, has a tetraloop configured to stabilize an adjacent stem region formed within the at least one strand.

As used herein, “non-naturally occurring” means an oligonucleotide, nucleic acid, peptide, polypeptide or protein that has been modified in a manner that would not otherwise exist in nature or is identical thereto but produced or derived via synthetic means (i.e., engineered, recombinant or modified by human manipulation).

As used herein, “nucleotide” means an organic compound having a nucleoside (a nucleobase such as, for example, adenine, cytosine, guanine, thymine, or uracil; and a pentose sugar such as, for example, ribose or 2′-deoxyribose) and a phosphate group. A “nucleotide” can serve as a monomeric unit of nucleic acids such as deoxyribonucleic acid (DNA) oligonucleotides and ribonucleic acid (RNA) oligonucleotides.

As used herein, “oligonucleotide” means a short nucleic acid (e.g., less than about 100 nucleotides in length). An oligonucleotide may be ss or ds. An oligonucleotide may or may not have duplex regions. Examples of oligonucleotides include, but are not limited to, an antisense oligonucleotide (ASO), a Dicer substrate interfering RNA (DsiRNA), a microRNA (miRNA), a short hairpin RNA (shRNA) and a small interfering RNA (siRNA).

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a ds oligonucleotide. In certain embodiments, the overhang is a 3′ or 5′ overhang on the antisense strand or sense strand of a ds oligonucleotides.

As used herein, “pharmaceutically acceptable buffer” means any of the standard pharmaceutical buffers known to one of skill in the art.

As used herein, “ribonucleotide” means a nucleotide having a ribose as its pentose sugar, which contains a hydroxyl group at its 2′ position. A modified ribonucleotide is a ribonucleotide having one or more modifications or substitutions of atoms other than hydrogen at the 2′ position, including modifications or substitutions in or of the nucleobase, sugar, or phosphate group.

As used herein, “iRNA,” “iRNA agent,” “RNAi,” “RNAi agent” and “RNA interference agent” means an oligonucleotide that contains RNA and that mediates the targeted cleavage of a RNA transcript via RNA interference, e.g., through a RNA-induced silencing complex (RISC) pathway. The RNAi agent can have a sense strand and an antisense strand, where the sense strand and the antisense strand form a duplex. In some instances, the sense and antisense strands of RNAi agent can be 21-23 nucleotides in length. Alternatively, the sense and antisense strands can be longer, for example, 25-36 nucleotides in length, in which case the longer nucleotide sequences are first processed by the Dicer enzyme. The RNAi agent directs sequence-specific degradation of mRNA via RNA interference. The RNAi agent attenuates, inhibits, modulates or reduces gene expression in a cell, tissue, organ, system or individual (here, e.g., ANGPTL3 or LPA expression).

As used herein, “strand” refers to a single, contiguous sequence of nucleotides linked together through internucleotide linkages (e.g., PO bonds/linkages or PS bond/linkages). A strand can have two free ends (e.g., a 5′ end and a 3′ end).

As used herein, “targeting ligand” means a chemical moiety that facilitates entry of an oligonucleotide such as a RNAi agent herein to a tissue or cell. It can be a compound (e g., amino sugar, carbohydrate, cholesterol, lipid or polypeptide) that selectively binds to a cognate compound (e.g., a receptor) of a tissue or cell of interest and that is conjugatable to another substance for targeting another substance to the tissue or cell of interest. For example, a targeting ligand may be conjugated to an oligonucleotide herein for purposes of targeting the oligonucleotide to a specific cell or tissue of interest. A targeting ligand can selectively bind to a cell surface receptor. Accordingly, a targeting ligand, when conjugated to an oligonucleotide, facilitates delivery of the oligonucleotide into a particular cell through selective binding to a receptor expressed on the surface of the cell and endosomal internalization by the cell of the complex comprising the oligonucleotide, targeting ligand, and receptor. Moreover, a targeting ligand can be conjugated to an oligonucleotide via a linker that is cleaved following or during cellular internalization such that the oligonucleotide is released from the targeting ligand in the cell.

### Compositions

### Oligonucleotide Fragments

The disclosure describes oligonucleotide fragments (i.e., intermediate compounds) having exemplary sequences/structures according to the following:

Intermediate Compound 1:
(SEQ ID NO: 5)
5 mUS-mU-mG-mC-mC-mA-mA-fG-fC-fU-fU-mG-mG-mU 3′.
Intermediate Compound 2:
(SEQ ID NO: 6)
5′ p-mC-mA-mU-mC-mU-mA-mG-mC-mA-mG 3′.
Intermediate Compound 3:
(SEQ ID NO: 7)
5′ p-mC-mC-mG-[ademA-GalNAc]-[ademA-GalNAc]-[ademA-GalNAc]-mG-mG-mC-mU-
mG-mC 3′.
Intermediate Compound 4:
(SEQ ID NO: 8)
5′ p-mA-mG-mC-fU-mU-mG-mG-mC-mA-mA^{S}-mG^{S}-mG 3′.
Intermediate Compound 5:
(SEQ ID NO: 9)
5′ [MePhosphonate-4O-mU^{S}]-fA^{S}-fG^{S}-fA-fU-mG-fA-mC-mC-fA 3′.
Intermediate Compound 6:
(SEQ ID NO: 10)
5′ p-mC-mA-mU-mC-mU-mA-mG-mC-mA-mG-mC-mC 3′
Intermediate Compound 7:
(SEQ ID NO: 11)
5′ p-mG-[ademA-GalNAc]-[ademA-GalNAc]-[ademA-GalNAc]-mG-mG-mC-mU-mG-mC 3′.
Intermediate Compound 8:
(SEQ ID NO: 12)
5′ p-mC-mA-mU-mC-mU-mA-mG-mC-mA-mG-mC 3′.
Intermediate Compound 9:
(SEQ ID NO: 13)
5′ p-mC-mG-[ademA-GalNAc]-[ademA-GalNAc]-[ademA-GalNAc]-mG-mG-mC-mU-mG-
mC 3′.
Intermediate Compound 10:
(SEQ ID NO: 14)
5′ mU^{S}-mU-mG-mC-mC-mA-mA-fG-fC-fU 3′.
Intermediate Compound 11:
(SEQ ID NO: 15)
5′ p-fU-mG-mG-mU-mC-mA-mU-mC-mU-mA-mG-mC-mA-mG 3′.
Intermediate Compound 12:
(SEQ ID NO: 16)
5′ p-fA-mC-mC-fA-mA-mG-mC-fU-mU-mG-mG-mC-mA-mA^{S}-mG^{S}-mG 3′.
Intermediate Compound 13:
(SEQ ID NO: 17)
5′ [MePhosphonate-4O-mU^{S}]-fA^{S}-fG^{S}-fA-fU-mG 3′.
Intermediate Compound 14:
(SEQ ID NO: 18)
5′ mU^{S}-mU-mG-mC-mC-mA-mA-fG-fC 3′.
Intermediate Compound 15:
(SEQ ID NO: 19)
5′ p-fU-fU-mG-mG-mU-mC-mA-mU-mC-mU-mA-mG-mC-mA-mG 3′.
Intermediate Compound 16:
(SEQ ID NO: 20)
5′ p-mC-mC-fA-mA-mG-mC-fU-mU-mG-mG-mC-mA-mA^{S}-mG^{S}-mG 3′.
Intermediate Compound 17:
(SEQ ID NO: 21)
5′ [MePhosphonate-4O-mU^{S}]-fA^{S}-fG^{S}-fA-fU-mG-fA 3′.
Intermediate Compound 18:
(SEQ ID NO: 22)
5′ mU^{S}-mU-mG-mC-mC-mA-mA-fG 3′.
Intermediate Compound 19:
(SEQ ID NO: 23)
5′ p-fC-fU-fU-mG-mG-mU-mC-mA-mU-mC-mU-mA-mG-mC-mA-mG 3′.
Intermediate Compound 20:
(SEQ ID NO: 24)
5′ p-mC-fA-mA-mG-mC-fU-mU-mG-mG-mC-mA-mA^{S}-mG^{S}-mG 3′.
Intermediate Compound 21:
(SEQ ID NO: 25)
5′ [MePhosphonate-4O-mU^{S}]-fA^{S}-fG^{S}-fA-fU-mG-fA-mC 3′.
Intermediate Compound 22:
(SEQ ID NO: 26)
5′ p-mC-mA-mU-mC-mU-mA-mG-mC-mA-mG-mC-mC-mG 3′.
Intermediate Compound 23:
(SEQ ID NO: 27)
5′ p-[ademA-GalNAc]-[ademA-GalNAc]-[ademA-GalNAc]-mG-mG-mC-mU-mG-mC 3′.
Intermediate Compound 24:
(SEQ ID NO: 28)
5′ p-mC-mA-mU-mC-mU-mA-mG-mC-mA-mG-mC-mC-mG-[ademA-GalNAc]-[ademA-
GalNAc]-[ademA-GalNAc]-mG-mG 3′.
Intermediate Compound 25:
(SEQ ID NO: 29)
5′ mC-mU-mG-mC 3′.
Intermediate Compound 26:
(SEQ ID NO: 30)
5′ p-mG-mC-mA-mA^{S}-mG^{S}-mG 3′.
Intermediate Compound 27:
(SEQ ID NO: 31)
5′ p-mA-mG-mC-fU-mU-mG 3′.
Intermediate Compound 28:
(SEQ ID NO: 32)
5′ mU^{S}-mC-mA-mA-mA-mA-mU-fG-fG-fA-fA-mG-mG-mU 3′.
Intermediate Compound 29:
(SEQ ID NO: 33)
5′ p-mU-mA-mU-mA-mC-mA-mG-mC-mA-mG 3′.
Intermediate Compound 30:
(SEQ ID NO: 34)
5′ p-mU-mC-mC-fA-mU-mU-mU-mU-mG-mA^{S}-mG^{S}-mG 3′.
Intermediate Compound 31:
(SEQ ID NO: 35)
5′ [MePhosphonate-4O-mU^{S}]-fG^{S}-fU^{S}-fA-fU-mA-fA-mC-mC-fU 3′.
Intermediate Compound 32:
(SEQ ID NO: 36)
5′ p-mC-mA-mU-mC-mU-mA-mG-mC-mA-mG-mC-mC-mG-[ademA-GalNAc]-[ademA-
GalNAc]-[ademA-GalNAc]-mG-mG-mC-mU-mG-mC 3′.
Intermediate Compound 33:
(SEQ ID NO: 37)
5′ cyclo-(mC-mA-mU-mC-mU-mA-mG-mC-mA-mG-mC-mC-mG-[ademA-GalNAc]-
[ademA-GalNAc]-[ademA-GalNAc]-mG-mG-mC-mU-mG-mC) 3′.

As noted above and detailed below, certain combinations of the oligonucleotide fragments herein can be ligated together and are useful in making a first RNAi agent having a sense strand of SEQ ID NO:1 and an antisense strand of SEQ ID NO:2. Other combinations of the oligonucleotide fragments herein can be ligated together and are useful in making a second RNAi agent having a sense strand of SEQ ID NO:3 and an antisense strand of SEQ ID NO:4.

### RNAi Agents

As noted above, certain combinations of the oligonucleotide fragments herein (i.e., intermediate compounds) can be ligated to form the RNAi agents herein.

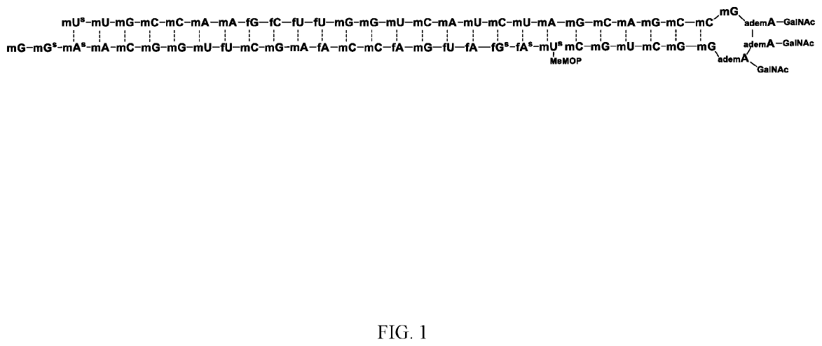

A first RNAi agent that can be formed from certain of the oligonucleotide fragments herein includes a sense strand having a nucleotide sequence of SEQ ID NO:1 and an antisense strand having a nucleotide sequence of SEQ ID NO:2, as shown in FIG. 1. The first RNAi agent is useful in attenuating, preventing and/or treating diseases, disorders and/or conditions associated with LPA expression.

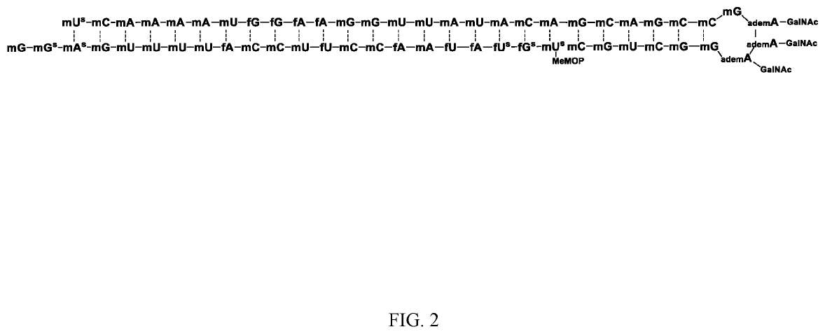

A second RNAi agent that can be formed from certain of the oligonucleotide fragments herein includes a sense strand having a nucleotide sequence of SEQ ID NO:3 and an antisense strand having a nucleotide sequence of SEQ ID NO:4, as shown in FIG. 2. The second RNAi agent is useful in attenuating, preventing and/or treating diseases, disorders and/or conditions associated with ANGPTL3 expression.

### Methods

The methods can include the steps described herein, and these maybe be, but not necessarily, carried out in the sequence as described. Other sequences, however, also are conceivable. Moreover, individual or multiple steps may be carried out either in parallel and/or overlapping in time and/or individually or in multiply repeated steps. Furthermore, the methods may include additional, unspecified steps.

### Standard Solid Phase Oligonucleotide Synthesis of Intermediate Compounds

The oligonucleotide fragments herein (i.e., intermediate compounds) can be made via standard oligonucleotide synthesis methods known in the art, such as SPOS. SPOS builds are accomplished using standard amidite chemistry techniques employing sequential coupling with automated oligonucleotide synthesizer. See, e.g., Paredes et al. (2018) Synthesis of Therapeutic Oligonucleotides, Springer Nature Singapore Pte Ltd., Paredes et al. (2017) Comprehensive Medicinal Chemistry III, pp. 233-279. Automated nucleic acid synthesizers, including DNA/RNA synthesizers, are commercially available from, for example, Applied Biosystems (Foster City, CA), BioAutomation (Irving, TX) and GE Healthcare Life Sciences (Pittsburgh, PA); see also, Intl. Patent Application Publication Nos. 2005/070859 and 2012/157723.

As one of skill in the art understands, other methods and/or techniques of synthesizing oligonucleotides may be used. Additionally, the various synthetic steps may be performed in an alternate sequence or order to give the desired compounds. Other synthetic chemistry transformations, protecting groups (e.g., for hydroxyl, amino, etc. present on the bases), and protecting group methodologies (protection and deprotection) useful in synthesizing the oligonucleotides are known in the art and are described in, for example, Larock, “Comprehensive Organic Transformations,” VCH Publishers (1989); Greene & Wuts, “Protective Groups in Organic Synthesis,” 2^{nd} Ed., John Wiley & Sons (1991); Fieser & Fieser, “Fieser & Fieser's Reagents for Organic Synthesis,” John Wiley & Sons (1994); and Paquette, ed., “Encyclopedia of Reagents for Organic Synthesis,” John Wiley & Sons (1995).

Briefly, during SPOS, nucleoside phosphoramidite building blocks can be added in successive cycles to a solid support to prepare an oligonucleotide of the desired length and sequence. Each cycle consists of several chemical reactions: detritylation, coupling, oxidation or thiolation and capping. After synthesis of a given oligonucleotide is complete, it is released from the solid support and protecting groups can be removed in the same step.

For detritylation, the initial resin can be swelled with ACN and then treated with 10% DCA in toluene.

The crude oligonucleotide fragments that are generated then can be purified generally using chromatographic purification to isolate full-length oligonucleotides from their related impurities. Finally, isolation through desalting and further lyophilization yields pure oligonucleotide solid material.

### Hybrid Solid Phase and Enzymatic Ligation to Form RNAi Agents

Certain combinations of the oligonucleotide fragments herein (i.e., intermediate compounds) prepared via SPOS as described above can be combined to obtain the RNAi agent of SEQ ID NOS:1 and 2 according to methods that are known to one of skill in the art. As such, the methods described herein can include synthesizing independent oligonucleotide fragments followed by ligating such fragments, thereby forming the RNAi agent of SEQ ID NOS:1 and 2.

For example, an exemplary method of making the oligonucleotide of SEQ ID NO:1 includes at least a step of ligating the following three (3) oligonucleotide fragments together, where such fragments have nucleotide sequences as recited in SEQ ID NOS:5, 6 and 7. In some instances, the fragments can be ligated in the following order: SEQ ID NO:5 to SEQ ID NO:6 to SEQ ID NO:7 (i.e., from 5′ end to 3 end′).

Alternatively, the oligonucleotide of SEQ ID NO:1 can be made by ligating the following three (3) oligonucleotide fragments together, where such fragments have nucleotide sequences as recited in SEQ ID NOS:5, 10 and 11. In some instances, the fragments can be ligated in the following order: SEQ ID NO:5 to SEQ ID NO:10 to SEQ ID NO:11 (i.e., from 5′ end to 3 end′).

Alternatively, the oligonucleotide of SEQ ID NO:1 can be made by ligating the following three (3) oligonucleotide fragments together, where such fragments have nucleotide sequences as recited in SEQ ID NOS:5, 12 and 13. In some instances, the fragments can be ligated in the following order: SEQ ID NO:5 to SEQ ID NO:12 to SEQ ID NO:13 (i.e., from 5′ end to 3 end′).

Alternatively, the oligonucleotide of SEQ ID NO:1 can be made by ligating the following three (3) oligonucleotide fragments together, where such fragments have nucleotide sequences as recited in SEQ ID NOS:7, 14 and 15. In some instances, the fragments can be ligated in the following order: SEQ ID NO:14 to SEQ ID NO:15 to SEQ ID NO:7 (i.e., from 5′ end to 3 end′).

Alternatively, the oligonucleotide of SEQ ID NO:1 can be made by ligating the following three (3) oligonucleotide fragments together, where such fragments have nucleotide sequences as recited in SEQ ID NOS:7, 18 and 19. In some instances, the fragments can be ligated in the following order: SEQ ID NO:18 to SEQ ID NO:19 to SEQ ID NO:7 (i.e., from 5′ end to 3 end′).

Alternatively, the oligonucleotide of SEQ ID NO:1 can be made by ligating the following three (3) oligonucleotide fragments together, where such fragments have nucleotide sequences as recited in SEQ ID NOS:7, 22 and 23. In some instances, the fragments can be ligated in the following order: SEQ ID NO:22 to SEQ ID NO:23 to SEQ ID NO:7 (i.e., from 5′ end to 3 end′).

Alternatively, the oligonucleotide of SEQ ID NO:1 can be made by ligating the following three (3) oligonucleotide fragments together, where such fragments have nucleotide sequences as recited in SEQ ID NOS:5, 26 and 27. In some instances, the fragments can be ligated in the following order: SEQ ID NO:5 to SEQ ID NO:26 to SEQ ID NO:27 (i.e., from 5′ end to 3 end′).

Likewise, an exemplary method of making the oligonucleotide of SEQ ID NO:2 includes at least a step of ligating the following two (2) oligonucleotide fragments together, where such fragments have nucleotide sequences as recited in SEQ ID NOS:8 and 9. In some instances, the fragments can be ligated in the following order: SEQ ID NO:9 to SEQ ID NO:8 (i.e., from 5′ end to 3 end′).

Alternatively, the oligonucleotide of SEQ ID NO:2 can be made by ligating the following two (2) oligonucleotide fragments together, where such fragments have nucleotide sequences as recited in SEQ ID NOS:16 and 17. In some instances, the fragments can be ligated in the following order: SEQ ID NO:17 to SEQ ID NO:16 (i.e., from 5′ end to 3 end′).

Alternatively, the oligonucleotide of SEQ ID NO:2 can be made by ligating the following two (2) oligonucleotide fragments together, where such fragments have nucleotide sequences as recited in SEQ ID NOS:20 and 21. In some instances, the fragments can be ligated in the following order: SEQ ID NO:21 to SEQ ID NO:20 (i.e., from 5′ end to 3 end′).

Alternatively, the oligonucleotide of SEQ ID NO:2 can be made by ligating the following two (2) oligonucleotide fragments together, where such fragments have nucleotide sequences as recited in SEQ ID NOS:24 and 25. In some instances, the fragments can be ligated in the following order: SEQ ID NO:25 to SEQ ID NO:24 (i.e., from 5′ end to 3 end′).

Moreover, certain combinations of the oligonucleotide fragments herein (i.e., intermediate compounds) prepared via SPOS as described above can be combined to obtain the RNAi agent of SEQ ID NOS:3 and 4 according to methods that are known to one of skill in the art. Briefly, the methods can include synthesizing independent oligonucleotide fragments followed by ligating such fragments, thereby forming the RNAi agent of SEQ ID NOS:3 and 4.

For example, an exemplary method of making the oligonucleotide of SEQ ID NO:3 includes at least a step of ligating the following three (3) oligonucleotide fragments together, where such fragments have nucleotide sequences as recited in SEQ ID NOS:7, 32 and 33. In some instances, the fragments can be ligated in the following order: SEQ ID NO:32 to SEQ ID NO:33 to SEQ ID NO:7 (i.e., from 5′ end to 3 end′).

Likewise, an exemplary method of making the oligonucleotide of SEQ ID NO:4 includes at least a step of ligating the following two (2) oligonucleotide fragments together, where such fragments have nucleotide sequences as recited in SEQ ID NOS:34 and 35. In some instances, the fragments can be ligated in the following order: SEQ ID NO:35 to SEQ ID NO:34 (i.e., from 5′ end to 3 end′).

In any of the methods above, the ligating can be carried out in an aqueous solution such as a reaction buffer. In some instances, the solution can be an acetate buffer, a carbonate buffer, a citrate buffer or a phosphate buffer such as a Tris buffer. In addition, the solution can have a pH from about 5 to about 9, about 6 to about 8, or about 7. In other instances, the pH can be about 5, about 6, about 7, about 8 or about 9.

In addition, the aqueous solution can include a cofactor (e.g., adenosine triphosphate (ATP) or nicotinamide adenine dinucleotide (NAD)) and a divalent metal salt (e.g., MgCl₂).

Moreover, the oligonucleotide fragments can be present in the aqueous solution at a concentration from about 1 μM to about 100,000 μM (100 mM). In some instances, the oligonucleotide fragment concentration can be from about 100 μM to about 90,000 μM, about 1,000 μM to about 80,000 μM, about 2,000 μM to about 70,000 μM, about 3,000 μM to about 60,000 μM, about 4,000 μM to about 50,000 μM, about 5,000 μM to about 40,000 μM, about 6,000 μM to about 30,000 μM, about 7,000 μM to about 20,000 μM, about 8,000 μM to about 10,000 μM, or about 9,000 μM. In other instances, the oligonucleotide fragment concentration can be from about 100 μM to about 200 μM, about 200 μM to about 300 μM, about 300 μM to about 400 μM, about 400 μM to about 500 μM, about 500 μM to about 600 μM, about 60 μM to about 700 μM, about 700 μM to about 800 μM, about 800 μM to about 900 μM, about 900 μM to about 1,000 μM, about 1,000 μM to about 2,000 μM, about 2,000 μM to about 3,000 μM, about 3,000 μM to about 4,000 μM, about 4,000 μM to about 5,000 μM, about 5,000 μM to about 6,000 μM, about 6,000 μM to about 7,000 μM, about 7,000 μM to about 8,000 μM, about 8,000 μM to about 9,000 μM, about 9,000 μM to about 10,000 μM, about 10,000 μM to about 20,000 μM, about 20,000 μM to about 30,000 μM, about 30,000 μM to about 40,000 μM, about 40,000 μM to about 50,000 μM, about 50,000 μM to about 60,000 μM, about 60,000 μM to about 70,000 μM, about 70,000 μM to about 80,000 μM, about 80,000 μM to about 90,000 μM, or about 90,000 μM to about 100,000 μM. In yet other instances, the oligonucleotide fragment concentration can be about 1 μM, about 10 μM, about 20 μM, about 30 μM, about 4 μM, about 50 μM, about 60 μM, about 70 μM, about 80 μM, about 90 μM, about 100 μM, about 200 μM, about 300 μM, about 400 μM, about 500 μM, about 600 μM, about 700 μM, about 800 μM, about 900 μM, about 1,000 μM, about 1,500 μM, about 2,000 μM, about 2,500 μM, about 3,000 μM, about 3,500 μM, about 4,000 μM, about 4,500 μM, about 5,000 μM, about 5,500 μM, about 6,000 μM, about 6,500 μM, about 7,000 μM, about 7,500 μM, about 8,000 μM, about 8,500 μM, about 9,000 μM, about 9,500 μM, about 10,000 μM, about 10,500 μM, about 11,000 μM, about 11,500, about 12,000 μM, about 12,500 μM, about 13,000 μM, about 13,500, about 14,000 μM, about 14,500, about 15,000 μM, about 15,500 μM, about 16,000 μM, about 16,500 μM, about 17,000 μM, about 17,500 μM, about 18,000 μM, about 18,500 μM, about 19,000 μM, about 19,500 μM, about 20,000 μM, about 25,000 μM, about 30,000 μM, about 35,000 μM, about 40,000 μM, about 45,000, about 50,000 μM, about 55,000 μM, about 60,000 μM, about 65,000 μM, about 70,000 μM, about 75,000 μM, about 80,000 μM, about 85,000 μM, about 90,000 μM, about 95,000 μM or about 100,000 μM. Moreover, in some instances, each oligonucleotide fragment can be at a same concentration as the other oligonucleotide fragments. In other instances, each oligonucleotide fragment can be at a different concentration from the other oligonucleotide fragments.

Furthermore, the ligating can be at a reaction temperature sufficient for activating the enzyme, which can be from about 2° C. to about 50° C. In some instances, the reaction temperature is from about 5° C. to about 45° C., about 10° C. to about 40° C., about 15° C. to about 35° C., about 20° C. to about 30° C., or about 25° C. In other instances, the reaction temperature is from about 5° C. to about 10° C., from about 10° C. to about 15° C., from about 15° C. to about 20° C., from about 20° C. to about 25° C., from about 25° C. to about 30° C., from about 30° C. to about 35° C., from about 35° C. to about 40° C., from about 40° C. to about 45° C., or from about 45° C. to about 50° C. In yet other instances, the reaction temperature is about 5° C., about 10° C., about 15° C., about 20° C., about 25° C., about 30° C., about 35° C., about 40° C., about 45° C. or about 50° C.

Likewise, the ligating can be for a reaction time sufficient to produce a target oligonucleotide, which can be for about 1 hr to about 72 hr. In some instances, the reaction time can be for about 2 hr to about 70 hr, about 4 hr to about 68 hr, about 6 hr to about 66 hr, about 8 hr to about 64 hr, about 10 hr to about 62 hr, about 12 hr to about 60 hr, about 14 hr to about 58 hr, about 16 hr to about 56 hr, about 18 hr to about 54 hr, about 20 hr to about 52 hr, about 22 hr to about 54 hr, about 24 hr to about 52 hr, about 26 hr to about 50 hr, about 28 hr to about 48 hr, about 30 hr to about 46 hr, about 32 hr to about 44 hr, about 34 hr to about 42 hr, about 36 hr to about 40 hr, or about 38 hr. In other instances, the reaction time can be for about 2 hr to about 10 hr, about 10 hr to about 20 hr, about 20 hr to about 30 hr, about 30 hr to about 40 hr, about 40 hr to about 50 hr, about 50 hr to about 60 hr, or about 60 hr to about 70 hr. In yet other instances, the reaction time can be for about 2 hr, about 4 hr, about 6 hr, about 8 hr, about 10 hr, about 12 hr, about 14 hr, about 16 hr, about 18 hr, about 20 hr, about 22 hr, about 24 hr, about 26 hr, about 28 hr, about 30 hr, about 32 hr, about 34 hr, about 36 hr, about 38 hr, about 40 hr, about 42 hr, about 44 hr, about 46 hr, about 48 hr, about 50 hr, about 52 hr, about 54 hr, about 56 hr, about 58 hr, about 60 hr, about 62 hr, about 64 hr, about 66 hr, about 68 hr, about 70 hr or about 72 hr.

In the methods above, the ligating step can be via an enzyme. In some instances, the enzyme is a ligase, such as a DNA ligase or a RNA ligase. In some instances, the ligase is a naturally occurring (i.e., wild type) ligase. In other instances, the ligase is a non-naturally occurring (i.e., modified) ligase. Examples of ligases that can be used in the methods include, but are not limited to, T4 RNA ligase 1 or T4 RNA ligase 2. In some instances, the ligase is a T4 RNA ligase 1. In other instances, the ligase is a T4 RNA ligase 2.

RNA ligases are commercially available from sources such as, for example, Ajinomoto, Almac, Codexis, New England Biolabs, Takara and ThermoFisher Scientific.

The enzyme activity can be from about 0.01 U/μL to about 1 U/μL. In some instances, the activity can be from about 0.05 U/μL to about 0.95 U/μL, about 0.1 U/μL to about 0.9 U/μL, about 0.15 U/μL to about 0.85 U/μL, about 0.2 U/μL to about 0.7 U/μL, about 0.25 U/μL to about 0.65 U/μL, about 0.3 U/μL to about 0.6 U/μL, about 0.35 U/μL to about 0.55 U/μL about 0.4 U/μL to about 0.5 U/μL, or about 0.45 U/μL. In other instances, the activity can be about 0.01 U/μL, about 0.02 U/μL, about 0.03 U/μL, about 0.04 U/μL, about 0.05 U/μL, about 0.06 U/μL, about 0.07 U/μL, about 0.08 U/μL, about 0.09 U/μL, about 0.1 U/μL, about 0.15 U/μL, about 0.2 U/μL, about 0.25 U/μL, about 0.3 U/μL, about 0.35 U/μL, about 0.4 U/μL, about 0.45 U/μL, about 0.5 U/μL, about 0.55 U/μL, about 0.6 U/μL, about 0.65 U/μL, about 0.7 U/μL, about 0.75 U/μL, about 0.8 U/μL, about 0.85 U/μL, about 0.9 U/μL, about 0.95 or about 1 U/μL.

Alternatively, the enzyme concentration can be from about 0.01 g/L to about 10 g/L. In some instances, the concentration can be from about 0.05 g/L to about 9.9 g/L, about 0.1 g/L to about 9.8 g/L, about 0.2 g/L to about 9.7 g/L, about 0.3 g/L to about 9.6 g/L, about 0.4 g/L to about 9.5 g/L, about 0.5 g/L to about 9.4 g/L, about 0.6 g/L to about 9.3 g/L, about 0.7 g/L to about 9.2 g/L, about 0.8 g/L to about 9.1 g/L, about 0.9 g/L to about 9 g/L, about 1 g/L to about 8.9 g/L, about 1.1 g/L to about 8.8 g/L, about 1.2 g/L to about 8.7 g/L, about 1.3 g/L to about 8.6 g/L, about 1.4 g/L to about 8.5 g/L, about 1.5 g/L to about 8.4 g/L, about 1.6 g/L to about 8.3 g/L, about 1.7 g/L to about 8.2 g/L, about 1.8 g/L to about 8.1 g/L, about 1.9 g/L to about 8 g/L, about 2 g/L to about 7.9 g/L, about 2.1 g/L to about 7.8 g/L, about 2.2 g/L to about 7.7 g/L, about 2.3 g/L to about 7.6 g/L, about 2.4 g/L to about 7.5 g/L, about 2.5 g/L to about 7.4 g/L, about 2.6 g/L to about 7.3 g/L, about 2.7 g/L to about 7.2 g/L, about 2.8 g/L to about 7.1 g/L, about 2.9 g/L to about 7 g/L, about 3 g/L to about 6.9 g/L, about 3.1 g/L to about 6.8 g/L, about 3.2 g/L to about 6.7, about 3.3 g/L to about 6.6 g/L, about 3.4 g/L to about 6.5 g/L, about 3.5 g/L to about 6.4 g/L, about 3.6 g/L to about 6.3 g/L, about 3.7 g/L to about 6.2 g/L, about 3.8 g/L to about 6.1 g/L, about 3.9 g/L to about 6 g/L, about 4 g/L to about 5.9 g/L, about 4.1 g/L to about 5.8 g/L, about 4.2 g/L to about 5.7 g/L, about 4.3 g/L to about 5.6 g/L, about 4.4 g/L to about 5.5 g/L, about 4.5 g/L to about 5.4 g/L, about 4.6 g/L to about 5.3, about 4.7 g/L to about 5.2 g/L, about 4.8 g/L to about 5.1 g/L, about 4.9 g/L to about 5 g/L. In other instances, the concentration can be about 0.01 g/L, about 0.02 g/L, about 0.03 g/L, about 0.04 g/L, about 0.05 g/L, about 0.1 g/L, about 0.2 g/L, about 0.3 g/L, about 0.4 g/L, about 0.5 g/L, about 0.6 g/L, about 0.7 g/L, about 0.8 g/L, about 0.9 g/L, about 1 g/L, about 1.1 g/L, about 1.2 g/L, about 1.3 g/L, about 1.4 g/L, about 1.5 g/L, about 1.6 g/L, about 1.7 g/L, about 1.8 g/L, about 1.9 g/L, about 2 g/L, about 2.1 g/L, about 2.2 g/L, about 2.3 g/L, about 2.4 g/L, about 2.5 g/L, about 2.6 g/L, about 2.7 g/L, about 2.8 g/L, about 2.9 g/L, about 3 g/L, about 3.1 g/L, about 3.2 g/L, about 3.3 g/L, about 3.4 g/L, about 3.5 g/L, about 3.6 g/L, about 3.7 g/L, about 3.8 g/L, about 3.9 g/L, about 4 g/L, about 4.1 g/L, about 4.2 g/L, about 4.3 g/L, about 4.4 g/L, about 4.5 g/L, about 4.6 g/L, about 4.7, about 4.8 g/L, about 4.9 g/L, about 5 g/L, about 5.1 g/L, about 5.2 g/L, about 5.3 g/L, about 5.4 g/L, about 5.5 g/L, about 5.6 g/L, about 5.7 g/L, about 5.8 g/L, about 5.9 g/L, about 6 g/L, about 6.1 g/L, about 6.2 g/L, about 6.3 g/L, about 6.4 g/L, about 6.5 g/L, about 6.6 g/L, about 6.7 g/L, about 6.8 g/L, about 6.9 g/L, about 7 g/L, about 7.1 g/L, about 7.2 g/L, about 7.3 g/L, about 7.4 g/L, about 7.5 g/L, about 7.6 g/L, about 7.7 g/L, about 7.8 g/L, about 7.9 g/L, about 8 g/L, about 8.1 g/L, about 8.2 g/L, about 8.3 g/L, about 8.4 g/L, about 8.5 g/L, about 8.6 g/L, about 8.7 g/L, about 8.8 g/L, about 8.9 g/L, about 9 g/L, about 9.1 g/L, about 9.2 g/L, about 9.3 g/L, about 9.4 g/L, about 9.5 g/L, about 9.6 g/L, about 9.7 g/L, about 9.8 g/L, about 9.9 g/L or about 10 g/L. In certain instances, the concentration be about 0.025 g/L, about 0.1 g/L, about 0.3 g/L or about 1 g/L.

The methods above also can include a step of annealing the sense strand and the antisense stand to form a RNAi agent. In some instances, SEQ ID NO:1 and SEQ ID NO:2 are annealed to form a RNAi agent that modulates LPA expression such that complementary nucleotides in each strand hybridize/base pair with one another according to methods that are known to one of skill in the art. In other instances, SEQ ID NO:3 and SEQ ID NO:4 are annealed to form a RNAi agent that modulates ANGPTL3 expression such that complementary nucleotides in each strand hybridize/base pair with one another according to methods that are known to one of skill in the art.

### Other Methods/Uses

The RNAi agents herein can be used in a number of therapeutic applications. For example, the RNAi agent of SEQ ID NOS:1 and 2 can be used in methods of attenuating, preventing and/or treating diseases, disorders and/or conditions associated with LPA expression, where such methods include at least a step of administering to an individual in need of such treatment an effective amount of the RNAi agent of SEQ ID NOS:1 and 2, or a pharmaceutically acceptable salt thereof.

Likewise, the RNAi agent of SEQ ID NOS:3 and 4 can be used in methods of attenuating, preventing and/or treating diseases, disorders and/or conditions associated with ANGPTL3 expression, where such methods include at least a step of administering to an individual in need of such treatment an effective amount of the RNAi agent of SEQ ID NOS:3 and 4, or a pharmaceutically acceptable salt thereof.

### EXAMPLES

The following non-limiting examples are offered for purposes of illustration, not limitation.

### Oligonucleotide Fragment Development and Synthesis

### Example 1: SPOS of Intermediate Compound 1

Synthesis: 5′ mU^{S}-mU-mG-mC-mC-mA-mA-fG-fC-fU-fU-mG-mG-mU 3′ (SEQ ID NO:5), or a pharmaceutically acceptable salt thereof, was synthesized by standard SPOS. NittoPhase® μL 2′-OMe U 250 polystyrene resin (246 μmol/g, ~750 mg, ~185 mol) was charged to a stainless-steel column (6.3 CV, dia. 20 mm), which was then installed on an AKTA OligoPilot® Plus 100 synthesizer. SPOS was performed using the conditions outlined in Table 1.

TABLE 1
Method Parameters for SPOS.

| Step | Parameter | Value |
|---|---|---|
| Column wash | ACN (mL) | 50.4 (8 CV) |
| Detritylation | detritylation reagent | 3% dichloroacetic acid (DCA) in |
| | | toluene (v/v) |
| | linear velocity | 200-300 cm/hr |
| | monitored by UV | endpoint: UV <200 mAU at 350 nm |
| | deblocking solution, average | 60-85 |
| | amount per cycle (mL) | |
| | ACN volume for deblock wash (mL) | 37.8 (6 CV) |
| Coupling | amidite concentration | 0.1M |
| | (M in ACN) | |
| | amidite equivalence (eq) | 2 |
| | activator reagent | 0.5M 5-(ethylthio)-1H-tetrazole |
| | | in ACN |
| | activator equivalence (eq) | 10 |
| | activator:amidite ratio | 5:1 |
| | coupling time, min | 10 |
| | ACN volume for coupling wash, (mL) | 25.2 (4 CV) |
| Oxidation | oxidization reagent | 0.05M Iodine in pyridine/water |
| | | (90/10 v/v) |
| | oxidization equivalence (eq) | 4 |
| | oxidization contact time (min) | 3.5 |
| | ACN volume for oxidization | 12.6 (2 CV) |
| | wash (mL) | |
| Sulfurization | sulfurization reagent | 0.2M xanthane hydride in |
| | | pyridine |
| | sulfurization contact time (min) | 5.50 |
| | sulfurization column volume | 2.0 |
| | ACN volume for sulfurization | 12.6 (2 CV) |
| | wash (mL) | |
| Capping | Capping solution A | 20% 1-methylimidazole in ACN |
| | | (v/v) |
| | Capping solution B | 1:1 mixture B1 and B2 |
| | Capping solution B1 | 40 vol % acetic anhydride in ACN |
| | Capping solution B2 | 60 vol % 2,6-lutidine in ACN |
| | Capping solution A | 3.15 |
| | (amount per cycle mL) | |
| | Capping solution B | 3.15 |
| | (amount per cycle, mL) | |
| | Capping contact time (min) | 0.8 |
| | ACN volume for capping wash (mL) | 18.9 (3 CV) |
| Final | See, detritylation conditions above | |
| Detritylation (if | | |
| applicable) | | |
| Cyanoethyl | DEA | 20% DEA in ACN |
| Deprotection | | (v/v) |
| (on column) | DEA contact time (min) | 10 |
| | DEA volume (mL) | 39 |
| | ACN volume for DEA wash (mL) | 7 |
| Column Wash | ACN (mL) | 50.4 (8 CV) |

TABLE 2
Reagents Used During SPOS.

| | Synthesis | |
|---|---|---|
| # | Operation | Reagent |
| 1 | Pre-Synthesis | ACN |
| | Wash | |
| 2 | Detritylation | 3% DCA in toluene |
| 3 | Wash | ACN |
| 4 | Coupling | phosphoramidites: 0.1M in ACN |
| | | Activator: 0.5M ETT in ACN |
| 5 | Wash | ACN |
| 6 | Oxidation | 50 mM iodine in 90% pyridine/10% H₂O (v/v) |
| 7 | Wash | ACN |
| 8 | Thiolation | 0.2M xanthane hydride in pyridine |
| 9 | Wash | ACN |
| 10 | Capping | 1:1 mix of Cap A and Cap B |
| | | Cap A: 20% 1-methyl imidazole in ACN (v/v) |
| | | Cap B: 20% acetic anhydride + 30% 2,6-lutidine |
| | | in ACN (v/v) |
| 11 | Wash | ACN |
| 12 | Final | 3% DCA/toluene |
| | Detritylation | |
| | (if applicable) | |
| 13 | Post Synthesis | 20% diethylamine/ACN |

Resin cleavage and isolation: After synthesis, nitrogen was passed through the resin-bound intermediate oligonucleotide fragment until a consistent mass was achieved. Intermediate Compound 1 was cleaved from the resin, and the nucleobases were globally deprotected using concentrated NH₄OH in H₂O at 38° C. for 18 hr. 20 mL NH₄OH/g resin was charged to a pressure relief reaction vial containing the dry resin (~1.5 g/lot). The spent resin was filtered and rinsed with 2×20 mL of 1:1 EtOH:H₂O. The filtrate was collected in a round bottom flask, and NH₃ was removed by rotary evaporation. Three lots of Intermediate Compound 1 were combined for downstream processing.

Tangential flow filtration (TFF): A PendoTECH TFF system was used to desalt Intermediate Compound 1 and to exchange the NH₃ salt for a Na salt. Two Pall T-series Cassettes with Omega PES membranes (0.1 m², 1 kDa MWCO) were used in series. The membranes were conditioned with H₂O (~5 L) prior to processing. Intermediate Compound 1 solution was concentrated to ~50 mL, and then diafiltered 10× using 0.5 μM NaCl solution (500 mL) to convert to sodium phosphates. Intermediate Compound 1 sodium salt was subjected to water diafiltration until the permeate conductivity is below 50 μS/cm. Intermediate Compound 1 sodium salt retentate was collected along with 3 water flushes of the membrane. Intermediate Compound 1 was subjected to lyophilization and was isolated as a crude oligo powder.

Analysis: A Water's Acquity UPLC system equipped with a tunable UV (TUV) detector was used to assess the purity of Intermediate Compound 1. The mobile phase, column, gradient and general parameters for chromatography are outlined in Tables 3 and 4.

TABLE 3
Mobile Phase and Column for Chromatography.

| | 10 mM DIPEA + | | | |
|---|---|---|---|---|
| Mobile Phase A | 100 mM HFIP in H₂O | Mobile Phase B | ACN | |
| Column | 2.1 × 50 mm, | Column Temp. | 80° | C. |
| | 1.7 μm BEH C18, | | | |
| | 130 Å | | | |
| Flow rate | 0.6 mL/min | UV | 260 | nm |

TABLE 4
Gradient Parameters for Chromatography.

| Time (min) | % A | % B |
|---|---|---|
| Initial | 99.0 | 1.0 |
| 25.00 | 75.7 | 24.3 |
| 25.10 | 0.0 | 100.0 |
| 27.00 | 0.0 | 100.0 |
| 27.10 | 99.0 | 1.0 |
| 30.00 | 99.0 | 1.0 |

Results: Intermediate Compound 1 (1.89 g, 90.21% by UPLC, expected exact mass=4585.706 Da, observed exact mass=4585.702 Da) was prepared as a crude sodium salt.

### Example 2: SPOS of Intermediate Compound 2

Synthesis: 5′ p-mC-mA-mU-mC-mU-mA-mG-mC-mA-mG 3′ (SEQ ID NO:6), or a pharmaceutically acceptable salt thereof, was synthesized by standard SPOS. NittoPhase® HL 2′-OMe G(iBu) 250 polystyrene resin (249 μmol/g, ~850 mg, ~212 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

Analysis: Analysis was performed as described in Example 1.

TFF: TFF was performed as described in Example 1.

Results: Intermediate Compound 2 (1.89 g, 95.87% by UPLC, expected exact mass=3362.594 Da, observed exact mass=3362.588 Da) was prepared as a crude sodium salt.

### Example 3: SPOS of Intermediate Compound 3

Synthesis: 5′ p-mC-mC-mG-[ademA-GalNAc]-[ademA-GalNAc]-[ademA-GalNAc]-mG-mG-mC-mU-mG-mC 3′ (SEQ ID NO:7), or a pharmaceutically acceptable salt thereof, was synthesized by standard SPOS. NittoPhase® HL 2′-OMe C(Ac) 250 polystyrene resin (257 μmol/g, ~800 mg, ~206 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 1.

Analysis: Analysis was performed as described in Example 1; however, the mobile phase, column and gradient are outlined in Table 5.

TABLE 5
Mobile Phase and Column for Chromatography.

| | 10 mM DIPEA + | | | |
|---|---|---|---|---|
| Mobile Phase A | 100 mM HFIP in H₂O | Mobile Phase B | ACN | |
| Column | 2.1 × 50 mm, | Column temp. | 55° | C. |
| | 1.7 μm BEH C18, | | | |
| | 130 Å | | | |
| Flow rate | 0.6 mL/min | UV | 260 | nm |

Results: Intermediate Compound 3 (2.54 g, 88.22% by UPLC, expected exact mass=5298.322 Da, observed exact mass=5298.318 Da) was prepared as a crude sodium salt.

### Example 4: SPOS of Intermediate Compound 4

Synthesis: 5′ p-mA-mG-mC-fU-mU-mG-mG-mC-mA-mA^{S}-mG^{S}-mG 3′ (SEQ ID NO:8), or a pharmaceutically acceptable salt thereof, was synthesized by standard SPOS. NittoPhase® HL 2′-OMe G(iBu) 250 polystyrene resin (249 μmol/g, ~800 mg, ~200 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 1.

Analysis: Analysis was performed as described in Example 3.

Results: Intermediate Compound 4 (2.00 g, 89.03% by UPLC, expected exact mass=4140.661 Da, observed exact mass=4140.652 Da) was prepared as a crude sodium salt.

### Example 5: SPOS of Intermediate Compound 5

Synthesis: 5′ [MePhosphonate-40-mU^{S}]-fA^{S}-fG^{S}-fA-fU-mG-fA-mC-mC-fA 3′ (SEQ ID NO:9), or a pharmaceutically acceptable salt thereof, was synthesized by standard SPOS. NittoPhase® HL 2′-Fluoro A(bz) 250 polystyrene resin (229 μmol/g, ~850 mg, ~195 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 1.

Analysis: Analysis was performed as described in Example 3.

Results: Intermediate Compound 5 (1.57 g, 92.84% by UPLC, expected exact mass=3376.432 Da, observed exact mass=3376.426 Da) was prepared as a crude sodium salt.

### Example 6: SPOS of Intermediate Compound 6

Synthesis: 5′ p-mC-mA-mU-mC-mU-mA-mG-mC-mA-mG-mC-mC 3′ (SEQ ID NO:10), or a pharmaceutically acceptable salt thereof, was synthesized by standard SPOS. NittoPhase® HL Unylinker 350 polystyrene resin (346 μmol/g, 0.8031 g, 277.9 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: A Millipore Cogent pScale system was used to desalt Intermediate Compound 6 and to exchange the NH₃ salt for a sodium salt. A Sartorius Hydrosart® membrane (0.02 m², 2 kDa MWCO) was equipped on the system. The membranes were conditioned with H₂O (~0.5 L) prior to processing. Intermediate Compound 6 solution was concentrated to ~20 mL, and then diafiltered 10× using 0.5 μM NaCl solution (200 mL) to convert to sodium phosphates. Intermediate Compound 6 sodium salt was subjected to water diafiltration until the permeate conductivity was below 50 μS/cm. Intermediate Compound 6 sodium salt retentate was collected along with three water flushes of the membrane. Intermediate Compound 6 was subjected to lyophilization, and the crude oligonucleotide powder was dissolved in milliQ water (13.22 mL, OD/mL=1678.7) to create a stock solution.

Analysis: Analysis was performed as described in Example 3.

Results: Intermediate Compound 6 (0.783 g by optical density, 94.65% by UPLC, expected exact mass=4000.708 Da, observed exact mass=4000.719 Da) was prepared as a crude sodium salt.

### Example 7: SPOS of Intermediate Compound 7

Synthesis: 5′ p-mG-[ademA-GalNAc]-[ademA-GalNAc]-[ademA-GalNAc]-mG-mG-mC-mU-mG-mC 3′ (SEQ ID NO:11), or a pharmaceutically acceptable salt thereof, was synthesized by standard SPOS. NittoPhase® HL Unylinker 350 polystyrene resin (346 μmol/g, 0.7503 g, 259.6 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 6.

Analysis: Analysis was performed as described in Example 3.

Results: Intermediate Compound 7 (0.819 g by optical density, 89.44% by UPLC, expected exact mass=4660.208 Da, observed exact mass=4660.209 Da) was prepared as a crude sodium salt.

### Example 8: SPOS of Intermediate Compound 8

Synthesis: 5′ p-mC-mA-mU-mC-mU-mA-mG-mC-mA-mG-mC 3′ (SEQ ID NO:12), or a pharmaceutically acceptable salt thereof, was synthesized by standard SPOS. NittoPhase® HL Unylinker 350 polystyrene resin (346 μmol/g, 0.8007 g, 277.0 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 6.

Analysis: Analysis was performed as described in Example 3.

Results: Intermediate Compound 8 (0.719 g by optical density, 95.15% by UPLC, expected exact mass=3681.651 Da, observed exact mass=3681.655 Da) was prepared as a crude sodium salt.

### Example 9: SPOS of Intermediate Compound 9

Synthesis: 5′ p-mC-mG-[ademA-GalNAc]-[ademA-GalNAc]-[ademA-GalNAc]-mG-mG-mC-mU-mG-mC 3′ (SEQ ID NO:13), or a pharmaceutically acceptable salt thereof, was synthesized by standard SPOS. NittoPhase® HL Unylinker 350 polystyrene resin (346 μmol/g, 0.7454 g, 257.9 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 6.

Analysis: Analysis was performed as described in Example 3.

Results: Intermediate Compound 9 (0.896 g by optical density, 90.24% by UPLC, expected exact mass=4979.265 Da, observed exact mass=4979.264 Da) was prepared as a crude sodium salt.

### Example 10: SPOS of Intermediate Compound 10

Synthesis: 5′ mU^{S}-mU-mG-mC-mC-mA-mA-fG-fC-fU 3′ (SEQ ID NO:14), or a pharmaceutically acceptable salt thereof, was synthesized by standard SPOS. NittoPhase® HL Unylinker 350 polystyrene resin (346 μmol/g, 0.8066 g, 279.1 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: A PendoTECH and Millipore Cogent pScale TFF system were both used to desalt Intermediate Compound 10 and to exchange the NH₃ salt for a Na salt. A Sartorius Hydrosart® membrane (0.02 m², 2 kDa MWCO) was equipped on the system. The membranes were conditioned with H₂O (~0.5 L) prior to processing. Intermediate Compound 10 solution was concentrated to ~30 mL and then diafiltered 10× using 0.5M NaCl solution (300 mL) to convert to sodium phosphates. Intermediate Compound 10 sodium salt was subjected to water diafiltration until the permeate conductivity was below 50 μS/cm. Intermediate Compound 10 sodium salt retentate was collected along with three water flushes of the membrane. Intermediate Compound 10 was subjected to lyophilization, and the crude oligo powder was dissolved in milliQ water (14.05 mL, OD/mL=1368.3) to create a stock solution.

Analysis: Analysis was performed as described in Example 3.

Results: Intermediate Compound 10 (0.655 g by optical density, 94.69% by UPLC, expected exact mass=3239.518 Da, observed exact mass=3239.522 Da) was prepared as a crude sodium salt.

### Example 11: SPOS of Intermediate Compound 11

Synthesis: 5′ p-fU-mG-mG-mU-mC-mA-mU-mC-mU-mA-mG-mC-mA-mG 3′ (SEQ ID NO:15), or a pharmaceutically acceptable salt thereof, was synthesized by standard SPOS. NittoPhase® HL Unylinker 350 polystyrene resin (346 μmol/g, 0.8010 g, 277.1 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 10.

Analysis: Analysis was performed as described in Example 3.

Results: Intermediate Compound 11 (0.930 g by optical density, 93.06% by UPLC, expected exact mass=4708.782 Da, observed exact mass=4708.791 Da) was prepared as a crude sodium salt.

### Example 12: SPOS of Intermediate Compound 12

Synthesis: 5′ p-fA-mC-mC-fA-mA-mG-mC-fU-mU-mG-mG-mC-mA-mA^{S}-mG^{S}-mG 3′ (SEQ ID NO:16), or a pharmaceutically acceptable salt thereof, was synthesized by standard SPOS. NittoPhase® HL Unylinker 350 polystyrene resin (346 μmol/g, 0.8027 g, 277.7 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 10.

Analysis: Analysis was performed as described in Example 3.

Results: Intermediate Compound 12 (0.899 g by optical density, 82.82% by UPLC, expected exact mass=5440.871 Da, observed exact mass=5440.883 Da) was prepared as a crude sodium salt.

### Example 13: SPOS of Intermediate Compound 13

Synthesis: 5′ [MePhosphonate-40-mU^{S}]-fA^{S}-fG^{S}-fA-fU-mG 3′ (SEQ ID NO:17), or a pharmaceutically acceptable salt thereof, was synthesized by standard SPOS. NittoPhase® HL Unylinker 350 polystyrene resin (346 μmol/g, 0.8036 g, 278.0 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 10.

Analysis: Analysis was performed as described in Example 3.

Results: Intermediate Compound 13 (0.388 g by optical density, 94.34% by UPLC, expected exact mass=2076.222 Da, observed exact mass=2076.226 Da) was prepared as a crude sodium salt.

### Example 14: SPOS of Intermediate Compound 14

Synthesis: 5′ mU^{S}-mU-mG-mC-mC-mA-mA-fG-fC 3′ (SEQ ID NO:18), or a pharmaceutically acceptable salt thereof, was synthesized by standard SPOS. NittoPhase® HL Unylinker 350 polystyrene resin (346 μmol/g, 0.8043 g, 278.3 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: After synthesis, nitrogen was passed through the resin-bound intermediate oligonucleotide fragment (1.7125 g, 3.26 g/mmol mass gain) until a consistent mass was achieved. Intermediate Compound 14 was cleaved from the resin, and the nucleobases were globally deprotected using concentrated NH₄OH in H₂O at 38° C. for 18 hr. 20 mL NH₄OH/g resin was charged to a pressure relief reaction vial containing the dry resin. The spent resin was filtered and rinsed with 2×5 mL of concentrated NH₄OH. The filtrate was collected in a 50-mL Falcon tube and was concentrated to dryness in a Genevac® EZ-2 Elite centrifugal evaporation system. Intermediate Compound 14 was reconstituted in nuclease-free water (15 mL, OD/mL=1076.7) to a desired concentration for subsequent use in an enzymatic ligation reaction.

Analysis: Analysis was performed as described in Example 3.

Results: Intermediate Compound 14 (0.388 g by optical density, 94.34% by UPLC, expected exact mass=2076.222 Da, observed exact mass=2076.226 Da) was prepared as a crude ammonium salt.

### Example 15: SPOS of Intermediate Compound 15

Synthesis: 5′ p-fU-fU-mG-mG-mU-mC-mA-mU-mC-mU-mA-mG-mC-mA-mG 3′ (SEQ ID NO:19), or a pharmaceutically acceptable salt thereof, was synthesized by standard SPOS. NittoPhase® HL Unylinker 350 polystyrene resin (346 μmol/g, 0.7019 g, 242.9 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 14.

Analysis: Analysis was performed as described in Example 3.

Results: Intermediate Compound 15 (0.824 g) by optical density, 90.12% by UPLC, expected exact mass=5016.803 Da, observed exact mass=5016.811 Da) was prepared as a crude ammonium salt.

### Example 16: SPOS of Intermediate Compound 16

Synthesis: 5′ p-mC-mC-fA-mA-mG-mC-fU-mU-mG-mG-mC-mA-mA^{S}-mG^{S}-mG 3′ (SEQ ID NO:20), or a pharmaceutically acceptable salt thereof, was synthesized by standard SPOS. NittoPhase® HL Unylinker 350 polystyrene resin (346 μmol/g, 0.7036 g, 243.4 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 14.

Analysis: Analysis was performed as described in Example 3.

Results: Intermediate Compound 16 (0.698 g by optical density, 90.09% by UPLC, expected exact mass=5109.823 Da, observed exact mass=5109.830 Da) was prepared as a crude ammonium salt.

### Example 17: SPOS of Intermediate Compound 17

Synthesis: 5′ [MePhosphonate-40-mU^{S}]-fA^{S}-fG^{S}-fA-fU-mG-fA 3′ (SEQ ID NO:21), or a pharmaceutically acceptable salt thereof, was synthesized by standard SPOS. NittoPhase® HL Unylinker 350 polystyrene resin (346 μmol/g, 0.8041 g, 278.2 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 14.

Analysis: Analysis was performed as described in Example 3.

Results: Intermediate Compound 17 (0.414 g by optical density, 93.23% by UPLC, expected exact mass=2407.270 Da, observed exact mass=2407.274 Da) was prepared as a crude ammonium salt.

### Example 18: SPOS of Intermediate Compound 18

Synthesis: 5′ mU^{S}-mU-mG-mC-mC-mA-mA-fG 3′ (SEQ ID NO:22), or a pharmaceutically acceptable salt thereof, was synthesized by standard SPOS. NittoPhase® HL Unylinker 350 polystyrene resin (346 μmol/g, 0.8010 g, 277.1 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 14.

Analysis: Analysis was performed as described in Example 3.

Results: Intermediate Compound 18 (0.536 g by optical density, 96.44% by UPLC, expected exact mass=2624.460 Da, observed exact mass=2624.463 Da) was prepared as a crude ammonium salt.

### Example 19: SPOS of Intermediate Compound 19

Synthesis: 5′ p-fC-fU-fU-mG-mG-mU-mC-mA-mU-mC-mU-mA-mG-mC-mA-mG 3′ (SEQ ID NO:23), or a pharmaceutically acceptable salt thereof, was synthesized by standard SPOS. NittoPhase® HL Unylinker 350 polystyrene resin (346 μmol/g, 0.6999 g, 242.2 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 14.

Analysis: Analysis was performed as described in Example 3.

Results: Intermediate Compound 19 (0.850 g by optical density, 88.94% by UPLC, expected exact mass=5323.840 Da, observed exact mass=5323.849 Da) was prepared as a crude ammonium salt.

### Example 20: SPOS of Intermediate Compound 20

Synthesis: 5′ p-mC-fA-mA-mG-mC-fU-mU-mG-mG-mC-mA-mA^{S}-mG^{S}-mG 3′ (SEQ ID NO:24), or a pharmaceutically acceptable salt thereof, was synthesized by standard SPOS. NittoPhase® HL Unylinker 350 polystyrene resin (346 μmol/g, 0.7008 g, 242.5 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 14.

Analysis: Analysis was performed as described in Example 3.

Results: Intermediate Compound 20 (0.685 g by optical density, 89.06% by UPLC, expected exact mass=4790.766 Da, observed exact mass=4790.771 Da) was prepared as a crude ammonium salt.

### Example 21: SPOS of Intermediate Compound 21

Synthesis: 5′ [MePhosphonate-40-mU^{S}]-fA^{S}-fG^{S}-fA-fU-mG-fA-mC 3′ (SEQ ID NO:25), or a pharmaceutically acceptable salt thereof, was synthesized by standard SPOS. NittoPhase® HL Unylinker 350 polystyrene resin (346 μmol/g, 0.8031 g, 277.9 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 14.

Analysis: Analysis was performed as described in Example 3.

Results: Intermediate Compound 21 (0.608 g by optical density, 92.67% by UPLC, expected exact mass=2726.327 Da, observed exact mass=2726.328 Da) was prepared as a crude ammonium salt.

### Example 22: SPOS of Intermediate Compound 22

Synthesis: 5′ p-mC-mA-mU-mC-mU-mA-mG-mC-mA-mG-mC-mC-mG 3′ (SEQ ID NO:26), or a pharmaceutically acceptable salt thereof, was synthesized by standard SPOS. NittoPhase® HL Unylinker 350 polystyrene resin (346 μmol/g, 0.8081 g, 279.6 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: A Millipore Cogent pScale TFF system was used to desalt Intermediate Compound 22 and to exchange the NH₄ salt for a Na salt. A Sartorius Hydrosart® membrane (0.02 m², 2 kDa MWCO) was equipped on the system. The membranes were conditioned with H₂O (~0.5 L) prior to processing. Intermediate Compound 22 solution was concentrated to ~40 mL and then diafiltered 10× using 0.5M NaCl solution (400 mL) to convert to sodium phosphates. Intermediate Compound 22 sodium salt is subjected to water diafiltration until the permeate conductivity was below 60 μS/cm. Intermediate Compound 22 sodium salt retentate is collected along with two water flushes of the membrane. Intermediate Compound 22 was subjected to lyophilization, and the crude oligo powder was dissolved in milliQ water (60.24 mL, OD/mL=415.07) to create a stock solution.

Analysis: Analysis was performed as described in Example 3.

Results: Intermediate Compound 22 (0.881 g by optical density, 94.11% by UPLC, expected exact mass=4359.771 Da, observed exact mass=4359.778 Da) was prepared as a crude sodium salt.

### Example 23: SPOS of Intermediate Compound 23

Synthesis: 5′ p-[ademA-GalNAc]-[ademA-GalNAc]-[ademA-GalNAc]-mG-mG-mC-mU-mG-mC 3′ (SEQ ID NO:27), or a pharmaceutically acceptable salt thereof, was synthesized by standard SPOS. NittoPhase® HL Unylinker 350 polystyrene resin (346 μmol/g, 0.8043 g, 278.3 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 22.

Analysis: Analysis was performed as described in Example 3.

Results: Intermediate Compound 23 (0.853 g by optical density, 86.25% by UPLC, expected exact mass=4301.145 Da, observed exact mass=4301.149 Da) was prepared as a crude sodium salt.

### Example 24: SPOS of Intermediate Compound 24

Synthesis: 5′ p-mC-mA-mU-mC-mU-mA-mG-mC-mA-mG-mC-mC-mG-[ademA-GalNAc]-[ademA-GalNAc]-[ademA-GalNAc]-mG-mG 3′ (SEQ ID NO:28), or a pharmaceutically acceptable salt thereof, was synthesized by standard SPOS. NittoPhase® HL 2′-OMe G(Ac) 250 polystyrene resin (247 μmol/g, ~650 mg, ~161 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1. In contrast to Example 1, only two lots were prepared in this example and combined prior to TFF.

TFF: TFF was performed as described in Example 22.

Analysis: Analysis was performed as described in Example 1.

Results: Intermediate Compound 24 (1.08 g, 94.38% by UPLC, expected exact mass=7325.687 Da, observed exact mass=7325.680 Da) was isolated as a crude sodium salt.

### Example 25: SPOS of Intermediate Compound 25

Synthesis: 5′ mC-mU-mG-mC 3′ (SEQ ID NO:29), or a pharmaceutically acceptable salt thereof, was synthesized by standard SPOS. Briefly, SPOS is conducted using preloaded resin (loading factor 0.250 mmol/g) with conditions set forth below in Tables 6 and 7.

TABLE 6
Method Parameters for SPOS.

| Step | Parameter | Value |
|---|---|---|
| Column wash | ACN | 4 CV |
| Detritylation | detritylation reagent | 10% DCA in toluene (v/v) |
| | linear velocity | 360 cm/hr |
| | monitored by UV | endpoint: UV <300 mAU at 350 nm |
| | deblocking solution, average | 1. After 1.0 CV initiate UV1 |
| | amount per cycle (mL) | watch at 350 nm |
| | | 2. Start UV1 integration at UV |
| | | trigger >300 mAU |
| | | 3. 1.5 CV after integration |
| | | start, UV1 watch at 350 nm |
| | | 4. End detritylation delivery at |
| | | UV trigger <300 mAU |
| | ACN volume for deblock wash | 4 CV |
| Coupling | amidite concentration | 0.2M |
| | (M in ACN) | |
| | amidite equivalence (eq) | 2 |
| | activator reagent | 0.6M 5-(ethylthio)-1H-tetrazole |
| | | in ACN |
| | activator equivalence (eq) | 9 |
| | activator:amidite ratio | 4.5:1 |
| | coupling time, min | 10 |
| | ACN volume for coupling wash, | 2 CV |
| Oxidation | oxidization reagent | 0.05M iodine in pyridine/water |
| | | (90/10 v/v) |
| | oxidization equivalence (eq) | 3 |
| | oxidization contact time (min) | 4.0 |
| | ACN volume for oxidization | 2 CV |
| | wash | |
| Sulfurization | sulfurization reagent | 0.2M xanthane hydride in |
| | | pyridine |
| | sulfurization contact time (min) | 6.0 |
| | sulfurization equivalence (eq) | 6.0 |
| | ACN volume for sulfurization | 2 CV |
| | wash | |
| Capping | Capping solution A | 20% 1-methylimidazole in ACN |
| | | (v/v) |
| | Capping solution B | 1:1 mixture B1 and B2 |
| | Capping solution B1 | 40 vol % acetic anhydride in ACN |
| | Capping solution B2 | 60 vol % 2,6-lutidine in ACN |
| | Capping solution A | 4.71 |
| | (amount per cycle mL) | |
| | Capping solution B | 4.71 |
| | (amount per cycle, mL) | |
| | Capping contact time (min) | 0.5 |
| | ACN volume for capping wash | 2 CV |
| Final | See, detritylation conditions above | |
| Detritylation (if | | |
| applicable) | | |
| Cyanoethyl | DEA | 20% DEA in ACN (v/v) |
| Deprotection | DEA contact time (min) | 10 |
| (on column) | DEA volume (mL) | 113 |
| | ACN volume for DEA wash | 4 CV |

TABLE 7
Reagents Used During SPOS.

| | Synthesis | |
|---|---|---|
| # | Operation | Reagent |
| 1 | Pre-Synthesis | ACN |
| | Wash | |
| 2 | Detritylation | 10% DEA in toluene |
| 3 | Wash | ACN |
| 4 | Coupling | phosphoramidites: 0.2M in ACN |
| | | Activator: 0.5M ETT in ACN |
| 5 | Wash | ACN |
| 6 | Oxidation | 50 mM iodine in 90% pyridine/10% H₂O (v/v) |
| 7 | Wash | ACN |
| 8 | Thiolation | 0.2M xanthane hydride in pyridine |
| 9 | Wash | ACN |
| 10 | Capping | 1:1 mix of Cap A and Cap B |
| | | Cap A: 20% 1-methyl imidazole in ACN (v/v) |
| | | Cap B: 20% acetic anhydride + 30% 2,6-lutidine |
| | | in ACN (v/v) |
| 11 | Wash | ACN |
| 12 | Final | 10% DCA/toluene |
| | Detritylation | |
| | (if applicable) | |
| 13 | Post Synthesis | 20% diethylamine/ACN |

TFF: TFF was performed as described in Example 22.

Analysis: Analysis was performed as described in Example 1.

Results: Intermediate Compound 25 (0.70 g, 96.7% by UPLC, expected exact mass=1335.228 Da) was isolated as a crude sodium salt.

### Example 26: SPOS of Intermediate Compound 26

Synthesis: 5′ p-mG-mC-mA-mA^{S}-mG^{S}-mG 3′ (SEQ ID NO:30), or a pharmaceutically acceptable salt thereof, was synthesized by standard SPOS as described in Example 25.

TFF: TFF was performed as described in Example 22.

Analysis: Analysis was performed as described in Example 1.

Results: Intermediate Compound 26 (0.45 g, 98.6% by UPLC, expected exact mass=2133.35 Da, observed exact mass=2133.31 Da) was isolated as a crude sodium salt.

### Example 27: SPOS of Intermediate Compound 27

Synthesis: 5′ p-mA-mG-mC-fU-mU-mG 3′ (SEQ ID NO:31), or a pharmaceutically acceptable salt thereof, was synthesized by standard SPOS as described in Example 25.

TFF: TFF was performed as described in Example 22.

Analysis: Analysis was performed as described in Example 1.

Results: Intermediate Compound 27 (0.85 g, 95.3% by UPLC, expected exact mass=2026.32 Da, observed exact mass=2026.29 Da) was isolated as a crude sodium salt.

### Example 28: SPOS of Intermediate Compound 28

Synthesis: 5′ mU^{S}-mC-mA-mA-mA-mA-mU-fG-fG-fA-fA-mG-mG-mU 3′ (SEQ ID NO:32), or a pharmaceutically acceptable salt thereof, was synthesized by standard SPOS. NittoPhase® HL Unylinker 350 polystyrene resin (346 μmol/g, ~750 mg, ~260 μmol) was charged to a stainless-steel column (6.3 CV, dia. 20 mm), which was then installed on an AKTA OligoPilot® Plus 100 synthesizer. SPOS was performed as described in Example 1. In contrast to Example 1, 2 lots were produced, and the resin from each lot was divided for 2 separate isolation methods.

Resin cleavage and isolation: After synthesis, nitrogen was passed through the resin-bound intermediate oligonucleotide fragment until a consistent mass was achieved. Intermediate Compound 32 was cleaved from the resin, and the nucleobases were globally deprotected using concentrated NH₄OH in H₂O at 38° C. for 18 hr. 20 mL NH₄OH/g resin was charged to a pressure relief reaction vial containing the dry resin.
Isolation Method A: The spent resin was filtered and rinsed with 2×20 mL of 1:1 EtOH:H₂O. The filtrate was collected in a round bottom flask, and the NH₃ was removed by rotary evaporation. The two lots of Intermediate Compound 28 were combined for downstream processing. A PendoTECH® TFF system was used to desalt Intermediate Compound 28 and to exchange the ammonium salt for a sodium salt. Two Pall T-series Cassettes with Omega PES membranes (0.1 m², 1 kDa MWCO) are used in series. The membranes were conditioned with H₂O prior to processing. Intermediate Compound 28 solution was concentrated to ~80 mL then diafiltered 7.5× using 0.5M NaCl solution (600 mL) to convert to sodium phosphates. Intermediate Compound 28 sodium salt is subjected to water diafiltration until the permeate conductivity was below 50 μS/cm. Intermediate Compound 28 sodium salt retentate was collected along with several water flushes (~300 mL total) of the membrane. Intermediate Compound 28 was subjected to lyophilization and isolated as a crude oligo powder.
Isolation Method B: The spent resin was filtered and rinsed with 2×5 mL of concentrated NH₄OH. The filtrate was collected in a 50-mL Falcon tube. The sample was concentrated to dryness in a Genevac EZ-2 Elite centrifugal evaporation system. Intermediate Compound 28 was reconstituted in nuclease-free water to a desired concentration for subsequent use in an enzymatic ligation reaction.

TFF: TFF was performed as described in Example 28—Isolation Method A.

Analysis: Analysis was performed as described in Example 1.

Results: Intermediate Compound 28 (1.08 g, 90.47% by UPLC, expected exact mass=4679.783 Da, observed exact mass=4679.776 Da) was isolated as a crude sodium salt.

### Example 29: SPOS of Intermediate Compound 29

Synthesis: 5′ p-mU-mA-mU-mA-mC-mA-mG-mC-mA-mG 3′ (SEQ ID NO:33), or a pharmaceutically acceptable salt thereof, was synthesized by standard SPOS. NittoPhase® HL Unylinker 350 polystyrene resin (346 μmol/g, ~850 mg, ~295 μmol) was charged to a stainless-steel column (6.3 CV, dia. 20 mm), which was then installed on an AKTA OligoPilot® Plus 100 synthesizer. SPOS was performed as described in Example 1. In contrast to Example 1, 2 lots were produced, and the resin from each lot was divided for 2 separate isolation methods as described in Example 28.

Resin cleavage and isolation: Resin cleavage and isolation was performed as described in Example 28.

TFF: TFF was performed as described in Example 22.

Analysis: Analysis was performed as described in Example 3.

Results: Intermediate Compound 29 (1.20 g, 95.74% by UPLC, expected exact mass=3386.605 Da, observed exact mass=3386.601 Da) was isolated as a crude sodium salt.

### Example 30: SPOS of Intermediate Compound 30

Synthesis: 5′ p-mU-mC-mC-fA-mU-mU-mU-mU-mG-mA^{S}-mG^{S}-mG 3′ (SEQ ID NO:34), or a pharmaceutically acceptable salt thereof, was synthesized by standard SPOS. NittoPhase® HL Unylinker 350 polystyrene resin (346 μmol/g, ~800 mg, ~277 μmol) was charged to a stainless-steel column (6.3 CV, dia. 20 mm), which was then installed on an AKTA OligoPilot® Plus 100 synthesizer. SPOS was performed as described in Example 1. In contrast to Example 1, 2 lots were produced, and the resin from each lot was divided for 2 separate isolation methods as described in Example 28.

Resin cleavage and isolation: Resin cleavage and isolation was performed as described in Example 28.

TFF: TFF was performed as described in Example 22.

Analysis: Analysis was performed as described in Example 3.

Results: Intermediate Compound 30 (1.10 g, 93.47% by UPLC, expected exact mass=4039.589 Da, observed exact mass=4039.584 Da) was isolated as a crude sodium salt.

### Example 31: SPOS of Intermediate Compound 31

Synthesis: 5′ [MePhosphonate-40-mU^{S}]-fG^{S}-fU^{S}-fA-fU-mA-fA-mC-mC-fU 3′ (SEQ ID NO:35), or a pharmaceutically acceptable salt thereof, was synthesized by standard SPOS. NittoPhase® HL Unylinker 350 polystyrene resin (346 μmol/g, ~800 mg, ~277 μmol) was charged to a stainless-steel column (6.3 CV, dia. 20 mm), which was then installed on an AKTA OligoPilot® Plus 100 synthesizer. SPOS was performed as described in Example 1. In contrast to Example 1, 2 lots were produced, and the resin from each lot was divided for 2 separate isolation methods as described in Example 28.

Resin cleavage and isolation: Resin cleavage and isolation was performed as described in Example 28.

TFF: TFF was performed as described in Example 22.

Analysis: Analysis was performed as described in Example 3.

Results: Intermediate Compound 31 (1.20 g, 89.24% by UPLC, expected exact mass=3314.383 Da, observed exact mass=3314.377 Da) was isolated as a crude sodium salt.

### Example 32: SPOS of Intermediate Compound 32

Synthesis: 5′ p-mC-mA-mU-mC-mU-mA-mG-mC-mA-mG-mC-mC-mG-[ademA-GalNAc]-[ademA-GalNAc]-[ademA-GalNAc]-mG-mG-mC-mU-mG-mC 3′ (SEQ ID NO:36), or a pharmaceutically acceptable salt thereof, or a pharmaceutically acceptable salt thereof, was synthesized by standard SPOS. NittoPhase® HL Unylinker 350 polystyrene resin (346 μmol/g, ~600 mg, ~208 μmol) was charged to a stainless-steel column (6.3 CV, dia. 20 mm), which was then installed on an AKTA OligoPilot® Plus 100 synthesizer. SPOS was performed as described in Example 1.

Resin cleavage and isolation: Resin cleavage and isolation was performed as described in Example 1.

TFF: TFF was performed as described in Example 22.

Analysis: Analysis was performed as described in Example 1.

Results: Intermediate Compound 32 (1.05 g by optical density, 71.55% by UPLC, expected exact mass=8642.905 Da, observed exact mass=8642.919 Da) was isolated as a crude sodium salt.

### Enzymatic Ligation of Oligonucleotide Fragments

### Example 33: Comparing Pure vs. Crude Oligonucleotide Fragments in an Enzymatic Ligation Catalyzed by RNA Ligase to Form an RNAi Agent

Method 1 (pure oligonucleotide fragments): A first RNAi agent having a sense strand of SEQ ID NO:1 and an antisense strand of SEQ ID NO:2 was synthesized using 1 g/L of a first RNA ligase (Almac) to ligate purified Intermediate Compounds 1, 2, 3, 4 and 5 (0.1 mM) in the presence of 2 mM ATP and MgCl2 (10 mM). A 10 mg/mL enzyme stock solution was prepared by dissolving the RNA ligase 1 in nuclease-free water. In a 2-mL HPLC vial, a reaction buffer (800 μl) was prepared by adding Tris-HCl, pH 7.5 (1000 mM, 40 μL), MgCl2 (100 mM, 80 μL), KCl (2000 mM, 40 μL), DTT (100 mM, 8 μL), ATP (10 mM, 160 μL), Intermediate Compound 1 (3.7 mM, 21.6 μL), Intermediate Compound 2 (4.7 mM, 17.0 μL), Intermediate Compound 3 (2.9 mM, 27.6 μl), Intermediate Compound 4 (4.5 mM, 17.8 μL), Intermediate Compound 5 (6.3 mM, 12.7 μL) and RNA ligase (10 mg/mL, 80 μL) in nuclease-free water (295.3 μL). The reaction mixture was thoroughly mixed by gently pipetting the solution up and down. The 2-mL HPLC vial was placed in an Eppendorf ThermoMixer® (500 rpm) at 35° C. for 3 hr. The reaction was quenched with EDTA (26.7 mM, 3 mL).

Method 2 (crude oligonucleotide fragments): Alternatively, the first RNAi agent was synthesized using 1 g/L of the first RNA ligase to ligate crude Intermediate Compounds 1, 2, 3, 4 and 5 (0.1 mM) in the presence of ATP (2 mM) and MgCl2 (10 mM). A 10 mg/mL enzyme stock solution was prepared by dissolving RNA ligase in nuclease-free water. In a 2-mL HPLC vial, a reaction buffer (800 μL) was prepared by adding Tris-HCl, pH 7.5 (1000 mM, 40 μL), MgCl2 (100 mM, 80 μL), KCl (2000 mM, 40 μL), DTT (100 mM, 8 μL), ATP (10 mM, 160 μL), Intermediate Compound 1 (4.5 mM, 17.8 μL), Intermediate Compound 2 (6.8 mM, 11.8 μL), Intermediate Compound 3 (3.6 mM, 22.2 μL), Intermediate Compound 4 (5.2 mM, 15.4 μL), Intermediate Compound 5 (6.4 mM, 12.5 μL) and RNA ligase (10 mg/mL, 80 μL) in nuclease-free water (312.3 μL). The reaction mixture was thoroughly mixed by gently pipetting the solution up and down. The 2-mL HPLC vial was placed in an Eppendorf ThermoMixer® (500 rpm) at 35° C. for 3 hr. The reaction was quenched with EDTA (26.7 mM, 3 mL).

Results: The ligation of Method 1 yielded 1.66 mg of the first RNAi agent (81.87% by UPLC), and the ligation of Method 2 yielded 1.66 mg of the first RNAi agent (83.44% by UPLC).

### Example 34: Comparing Pure vs. Crude Oligonucleotide Fragments in an Enzymatic Ligation Catalyzed by RNA Ligase to Form an RNAi Agent

Method 1 (pure oligonucleotide fragments): A first RNAi agent having a sense strand of SEQ ID NO:1 and an antisense strand of SEQ ID NO:2 was synthesized using 0.025 g/L of a second RNA ligase (Codexis) to ligate purified Intermediate Compounds 1, 2, 3, 4 and 5 (0.1 mM) in the presence of ATP (0.4 mM) and MgCl₂ (2.0 mM). In a 2-mL HPLC vial, a reaction buffer (800 μL) was prepared by adding Tris-HCl, pH 7.5 (1000 mM, 40 μL), MgCl₂ (100 mM, 20 μL), DTT (100 mM, 10.4 μL), ATP (10 mM, 40 μL), Intermediate Compound 1 (5.4 mM, 14.8 μL), Intermediate Compound 2 (7.4 mM, 10.8 μL), Intermediate Compound 3 (4.7 mM, 17.0 μL), Intermediate Compound 4 (6.0 mM, 13.3 μL) and Intermediate Compound 5 (7.5 mM, 10.8 μL) in nuclease-free water (622.9 μL). An enzyme working solution (0.125 g/L, 224 μL) was prepared by diluting RNA ligase (3.5 g/L, 8 μL) in an enzyme storage buffer (216 μL). The enzyme working solution (200 μL) was added to the reaction buffer (800 μl). The reaction mixture was thoroughly mixed by gently pipetting the solution up and down. The 2-mL HPLC vial was placed in an Eppendorf ThermoMixer® (500 rpm) at 37° C. for 2 hr. The reaction was quenched with EDTA (26.7 mM, 3 mL).

Method 2 (crude oligonucleotide fragments): Alternatively, the first RNAi agent was synthesized using 0.025 g/L the second RNA ligase to catalyze the ligation of crude Intermediate Compounds 1, 2, 3, 4 and 5 (0.1 mM) in the presence of ATP (0.4 mM) and MgCl₂ (2.0 mM). In a 2-mL HPLC vial, a reaction buffer (800 μL) was prepared by adding Tris-HCl, pH 7.5 (1000 mM, 40 μL), MgCl₂ (100 mM, 20 μL), DTT (100 mM, 10.4 μL), ATP (10 mM, 40 μL), Intermediate Compound 1 (5.4 mM, 14.8 μL), Intermediate Compound 2 (7.4 mM, 10.8 μL), Intermediate Compound 3 (4.7 mM, 17.0 μL), Intermediate Compound 4 (6.0 mM, 13.3 μL) and Intermediate Compound 5 (7.5 mM, 10.8 μL) in nuclease-free water (622.9 μL). An enzyme working solution (0.125 g/L, 224 μL) was prepared by diluting RNA ligase (3.5 g/L, 8 μl) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 2 hr. The reaction was quenched with EDTA (26.7 mM, 3 mL).

Results: The ligation of Method 1 yielded 1.66 mg of the first RNAi agent (89.43% by UPLC), and the ligation of Method 2 yielded 1.66 mg of the first RNAi agent (92.58% by UPLC).

### Example 35: Standard Conditions for Enzymatic Ligations Using RNA Ligase to Form an RNAi Agent

Synthesis: A first RNAi agent having a sense strand of SEQ ID NO:1 and an antisense strand of SEQ ID NO:2 was synthesized using 0.1 g/L of the first RNA ligase (Almac) to ligate Intermediate Compounds 1, 2, 3, 4 and 5 (0.4 mM) in the presence of ATP (2 mM) and MgCl₂ (10 mM). A 10 mg/mL enzyme stock solution was prepared by dissolving RNA ligase in nuclease-free water. In a 2-mL HPLC vial, a reaction buffer (1000 μL) was prepared by adding Tris-HCl, pH 7.5 (1000 mM, 50 μL), MgCl₂ (100 mM, 100 μL), KCl (2000 mM, 50 μL), DTT (100 mM, 10 μL), ATP (10 mM, 200 μL), Intermediate Compound 1 (4.5 mM, 88.9 μL), Intermediate Compound 2 (6.8 mM, 58.8 μL), Intermediate Compound 3 (3.6 mM, 111.1 μL), Intermediate Compound 4 (5.2 mM, 76.9 μL), Intermediate Compound 5 (6.4 mM, 62.5 μL) and RNA ligase (10 mg/mL, 10 μL) in nuclease-free water (181.8 μL). The reaction mixture was thoroughly mixed by gently pipetting the solution up and down. The 2-mL HPLC vial was placed in an Eppendorf ThermoMixer® (500 rpm) at 35° C. for 24 hr. The reaction was quenched with EDTA (26.7 mM, 3 mL).

Results: The ligation yielded 8.29 mg of the first RNAi agent (91.63% by UPLC).

### Example 36: Standard Conditions for Enzymatic Ligations Using RNA Ligase to Form an RNAi Agent

Synthesis: A first RNAi agent having a sense strand of SEQ ID NO:1 and an antisense strand of SEQ ID NO:2 was synthesized using 0.025 g/L the second RNA ligase (Codexis) to ligate Intermediate Compounds 1, 2, 3, 16 and 17 (0.4 mM) in the presence of ATP (1.5 mM) and MgCl₂ (3.0 mM). In a 2-mL HPLC vial, a reaction buffer (800 μL) was prepared by adding Tris-HCl, pH 7.5 (1000 mM, 40 μL), MgCl₂ (100 mM, 30 μL), DTT (100 mM, 10.4 μL), ATP (10 mM, 150.4 μL), Intermediate Compound 1 (4.5 mM, 88.9 μL), Intermediate Compound 2 (6.8 mM, 58.8 μL), Intermediate Compound 3 (3.6 mM, 111.1 μL), Intermediate Compound 4 (5.2 mM, 76.9 μL) and Intermediate Compound 5 (6.4 mM, 62.5 μL) in nuclease-free water (171.0 μL). An enzyme working solution (0.125 g/L, 224 μL) was prepared by diluting RNA ligase (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 24 hr. The reaction was quenched with EDTA (26.7 mM, 3 mL).

Results: This ligation yielded 8.29 mg of the first RNAi agent (90.24% by UPLC).

### Example 37: Enzymatic Ligation Using RNA Ligase to Form a RNAi Agent

Synthesis: A first RNAi agent having a sense strand of SEQ ID NO:1 and an antisense strand of SEQ ID NO:2 was synthesized using 0.025 g/L of the second RNA ligase (Codexis) to ligate Intermediate Compounds 1, 6, 7, 5 and 5 (0.4 mM) in the presence of ATP (1.5 mM) and MgCl₂ (3.0 mM). In a 2-mL HPLC vial, a reaction buffer (800 μL) was prepared by adding Tris-HCl, pH 7.5 (1000 mM, 40 μL), MgCl₂ (100 mM, 30 μL), DTT (100 mM, 10.4 μL), ATP (10 mM, 150.4 μL), Intermediate Compound 1 (4.5 mM, 88.9 μL), Intermediate Compound 6 (16.1 mM, 24.8 μL), Intermediate Compound 7 (15.1 mM, 26.5 μL), Intermediate Compound 4 (5.9 mM, 67.8 μL) and Intermediate Compound 5 (6.4 mM, 62.5 μL) in nuclease-free water (298.7 μL). An enzyme working solution (0.125 g/L, 224 μL) was prepared by diluting RNA ligase (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 23 hr. The reaction was quenched with EDTA (26.7 mM, 3 mL).

Results: The ligation yielded 8.29 mg of the first RNAi agent (87.34% by UPLC).

### Example 38: Enzymatic Ligation Using RNA Ligase to Form a RNAi Agent

Synthesis: A first RNAi agent having a sense strand of SEQ ID NO:1 and an antisense strand of SEQ ID NO:2 was synthesized using 0.025 g/L the second RNA ligase (Codexis) to ligate Intermediate Compounds 1, 8, 9, 4 and 5 (0.4 mM) in the presence of ATP (1.5 mM) and MgCl₂ (3.0 mM). In a 2-mL HPLC vial, a reaction buffer (800 μL) was prepared by adding Tris-HCl, pH 7.5 (1000 mM, 40 μL), MgCl₂ (100 mM, 30 μL), DTT (100 mM, 10.4 μL), ATP (10 mM, 150.4 μL), Intermediate Compound 1 (4.5 mM, 88.9 μL), Intermediate Compound 8 (21.3 mM, 18.8 μL), Intermediate Compound 9 (17.2 mM, 23.3 μL), Intermediate Compound 4 (5.9 mM, 67.8 μL) and Intermediate Compound 5 (6.4 mM, 62.5 μL) in nuclease-free water (307.9 μL). An enzyme working solution (0.125 g/L, 224 μL) was prepared by diluting RNA ligase (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 23 hr. The reaction was quenched with EDTA (26.7 mM, 3 mL).

Results: The ligation yielded 8.29 mg of the first RNAi agent (88.80% by UPLC).

### Example 39: Enzymatic Ligation Using RNA Ligase to Form a RNAi Agent

Synthesis: A first RNAi agent having a sense strand of SEQ ID NO:1 and an antisense strand of SEQ ID NO:2 was synthesized using 0.025 g/L the second RNA ligase (Codexis) to catalyze the ligation of Intermediate Compounds 10, 11, 3, 12 and 13 (0.4 mM) in the presence of ATP (1.5 mM) and MgCl₂ (3.0 mM). In a 2-mL HPLC vial, a reaction buffer (800 μL) was prepared by adding Tris-HCl, pH 7.5 (1000 mM, 40 μL), MgCl₂ (100 mM, 30 μL), DTT (100 mM, 10.4 μL), ATP (10 mM, 150.4 μL), Intermediate Compound 10 (15.1 mM, 26.5 μL), Intermediate Compound 11 (12.7 mM, 31.5 μL), Intermediate Compound 3 (3.1 mM, 129.0 μL), Intermediate Compound 12 (9.2 mM, 43.5 μL) and Intermediate Compound 13 (10.0 mM, 40.0 μL) in nuclease-free water (298.7 μL). An enzyme working solution (0.125 g/L, 224 μL) was prepared by diluting RNA ligase (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 24 hr. The reaction was quenched with EDTA (26.7 mM, 3 mL).

Results: The ligation yielded 8.29 mg of the first RNAi agent (78.71% by UPLC).

### Example 40: Enzymatic Ligation Using RNA Ligase to Form a RNAi Agent

Synthesis A first RNAi agent having a sense strand of SEQ ID NO:1 and an antisense strand of SEQ ID NO:2 was synthesized using 0.025 g/L the second RNA ligase (Codexis) to ligate Intermediate Compounds 14, 15, 3, 16 and 17 (0.4 mM) in the presence of ATP (1.5 mM) and MgCl₂ (3.0 mM). In a 2-mL HPLC vial, a reaction buffer (800 μL) was prepared by adding Tris-HCl, pH 7.5 (1000 mM, 40 μL), MgCl₂ (100 mM, 30 μL), DTT (100 mM, 10.4 μL), ATP (10 mM, 150.4 μL), Intermediate Compound 14 (13.3 mM, 30.1 μL), Intermediate Compound 15 (10.7 mM, 37.4 μL), Intermediate Compound 3 (3.1 mM, 129.0 μL), Intermediate Compound 16 (9.3 mM, 43.0 μL) and Intermediate Compound 17 (11.6 mM, 34.5 μL) in nuclease-free water (295.2 μL). An enzyme working solution (0.125 g/L, 224 μL) was prepared by diluting RNA ligase (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 24 hr. The reaction was quenched with EDTA (26.7 mM, 3 mL).

Results: The ligation yielded 8.29 mg of the first RNAi agent (90.44% by UPLC).

### Example 41: Enzymatic Ligation Using RNA Ligase to Form a RNAi Agent

Synthesis: A first RNAi agent having a sense strand of SEQ ID NO:1 and an antisense strand of SEQ ID NO:2 was synthesized 0.025 g/L the second RNA ligase (Codexis) to ligate Intermediate Compounds 18, 19, 3, 20 and 21 (0.4 mM) in the presence of ATP (1.5 mM) and MgCl₂ (3.0 mM). In a 2-mL HPLC vial, a reaction buffer (800 μL) was prepared by adding Tris-HCl, pH 7.5 (1000 mM, 40 μL), MgCl₂ (100 mM, 30 μL), DTT (100 mM, 10.4 μL), ATP (10 mM, 150.4 μL), Intermediate Compound 18 (14.4 mM, 27.8 μL), Intermediate Compound 19 (10.5 mM, 38.1 μL), Intermediate Compound 3 (3.1 mM, 129.0 μL), Intermediate Compound 20 (9.8 mM, 40.8 μL) and Intermediate Compound 21 (15.2 mM, 26.3 μL) in nuclease-free water (307.2 μL). An enzyme working solution (0.125 g/L, 224 μL) was prepared by diluting RNA ligase (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 24 hr. The reaction was quenched with EDTA (26.7 mM, 3 mL).

Results: The ligation yielded 8.29 mg of the first RNAi agent (89.18% by UPLC).

### Example 42: Enzymatic Ligation Using RNA Ligase to Form a RNAi Agent

Synthesis: A first RNAi agent having a sense strand of SEQ ID NO:1 and an antisense strand of SEQ ID NO:2 was synthesized 0.025 g/L of the second RNA ligase (Codexis) to ligate Intermediate Compounds 1, 22, 23, 4 and 5 (0.4 mM) in the presence of ATP (1.5 mM) and MgCl₂ (3.0 mM). In a 2-mL HPLC vial, a reaction buffer (800 μL) was prepared by adding Tris-HCl, pH 7.5 (1000 mM, 40 μL), MgCl₂ (100 mM, 30 μL), DTT (100 mM, 10.4 μL), ATP (10 mM, 150.4 μL), Intermediate Compound 1 (4.1 mM, 97.6 μL), Intermediate Compound 22 (3.3 mM, 121.2 μL), Intermediate Compound 23 (2.0 mM, 200.0 μL), Intermediate Compound 4 (5.5 mM, 72.7 μL) and Intermediate Compound 5 (6.2 mM, 64.5 μL) in nuclease-free water (13.2 μL). An enzyme working solution (0.125 g/L, 224 μL) was prepared by diluting RNA ligase 2 (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 23 hr. The reaction was quenched with EDTA (26.7 mM, 3 mL).

Results: The ligation reaction failed because the ligation between Intermediate Compound 22 and Intermediate Compound 23 did not occur; therefore, the ligation reaction failed to produce the desired sense strand of SEQ ID NO:1.

### Example 43: Enzymatic Ligation Using RNA Ligase to Form a RNAi Agent

Synthesis: A second RNAi agent having a sense strand of SEQ ID NO:3 and an antisense strand of SEQ ID NO:4 was synthesized using 0.4 g/L the first RNA ligase (Almac) to ligate crude Intermediate Compounds 28, 29, 3, 30 and 31 (0.4 mM) in the presence of ATP (2 mM) and MgCl₂ (10 mM). A 10 mg/mL enzyme stock solution was prepared by dissolving RNA ligase in nuclease-free water. In a 2-mL HPLC vial, a reaction buffer (1000 μL) was prepared by adding Tris-HCl, pH 7.5 (1000 mM, 50 μL), MgCl₂ (100 mM, 100 μL), KCl (2000 mM, 50 μL), DTT (100 mM, 10 μL), ATP (10 mM, 200 μL), Intermediate Compound 28 (4.4 mM, 90.9 μL), Intermediate Compound 29 (6.3 mM, 63.5 μL), Intermediate Compound 3 (3.5 mM, 114.3 μL), Intermediate Compound 30 (4.8 mM, 83.3 μL), Intermediate Compound 31 (6.4 mM, 62.5 μL) and RNA ligase (10 mg/mL, 30 μL) in nuclease-free water (145.5 μL). The reaction mixture was thoroughly mixed by gently pipetting the solution up and down. The 2-mL HPLC vial was placed in an Eppendorf ThermoMixer® (500 rpm) at 35° C. for 19 hr. The reaction was quenched with EDTA (26.7 mM, 3 mL).

Results: The ligation yielded 8.29 mg of the second RNAi agent (84.93% by UPLC).

### Example 44: Enzymatic Ligation Using RNA Ligase to Form a RNAi Agent

Synthesis: A second RNAi agent having a sense strand of SEQ ID NO:3 and an antisense strand of SEQ ID NO:4 was synthesized using 0.025 g/L of the second RNA ligase (Codexis) to ligate Intermediate Compounds 28, 29, 3, 30 and 31 (0.4 mM) in the presence of ATP (1.5 mM) and MgCl₂ (3.0 mM). In a 2-mL HPLC vial, a reaction buffer (800 μL) was prepared by adding Tris-HCl, pH 7.5 (1000 mM, 40 μL), MgCl₂ (100 mM, 30 μL), DTT (100 mM, 10.4 μL), ATP (10 mM, 150.4 μL), Intermediate Compound 28 (4.4 mM, 90.9 μL), Intermediate Compound 29 (6.3 mM, 63.5 μL), Intermediate Compound 3 (3.5 mM, 114.3 μL), Intermediate Compound 30 (4.8 mM, 83.3 μL) and Intermediate Compound 31 (6.4 mM, 62.5 μL) in nuclease-free water (154.7 μL). An enzyme working solution (0.125 g/L, 224 mL) was prepared by diluting RNA ligase (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 20 hr. The reaction was quenched with EDTA (26.7 mM, 3 mL).

Results: The ligation yielded 8.29 mg of the second RNAi agent (82.91% by UPLC).

### Example 45: Enzymatic Ligation Using RNA Ligase to Form Intermediate Compound 33

Synthesis: Intermediate Compound 33 (SEQ ID NO:37) was synthesized using 0.025 g/L the second RNA ligase (Codexis) to ligate Intermediate Compounds 2 and 3 (0.08 mM) in the presence of ATP (0.4 mM) and MgCl₂ (2.0 mM). In a 2-mL HPLC vial, a reaction buffer (800 μL) was prepared by adding Tris-HCl, pH 7.5 (1000 mM, 40 μL), MgCl₂ (100 mM, 20 μL), DTT (100 mM, 10.4 μL), ATP (10 mM, 40 μL), Intermediate Compound 2 (6.8 mM, 11.8 μL) and Intermediate Compound 3 (3.6 mM, 22.2 μL) in nuclease-free water (655.6 μL). An enzyme working solution (0.125 g/L, 224 μL) was prepared by diluting RNA ligase 2 (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 2 hr. The reaction was quenched with EDTA (26.7 mM, 3 mL).

Results: The ligation yielded 0.69 mg of Intermediate Compound 33 (94.58% by UPLC).

### Example 46: Suppressing Formation of Intermediate Compound 33 During Enzymatic Ligation Using RNA Ligase When Forming a RNAi Agent

Synthesis: A second RNAi agent having a sense strand of SEQ ID NO:3 and an antisense strand of SEQ ID NO:4 was synthesized using 0.025 g/L the second RNA ligase (Codexis) to ligate Intermediate Compounds 28, 29, 3, 30 and 31 (0.4 mM) in the presence of ATP (1.5 mM) and MgCl₂ (3.0 mM). In a 2-mL HPLC vial, a reaction buffer (685.7 μL) was prepared by adding Tris-HCl, pH 7.5 (1000 mM, 40 μL), MgCl₂ (100 mM, 30 μL), DTT (100 mM, 10.4 μL), ATP (10 mM, 150.4 μL), Intermediate Compound 28 (4.4 mM, 90.9 μL), Intermediate Compound 29 (6.3 mM, 63.5 μL), Intermediate Compound 30 (4.8 mM, 83.3 μL) and Intermediate Compound 31 (6.4 mM, 62.5 μL) in nuclease-free water (154.7 μL). An enzyme working solution (0.125 g/L, 224 μL) was prepared by diluting RNA ligase (3.5 g/L, 8 μL) in an enzyme storage buffer (216 μL). The enzyme working solution (200 μL) was added to the reaction buffer (800 μL). The reaction mixture was thoroughly mixed by gently pipetting the solution up and down. The 2-mL HPLC vial was placed in an Eppendorf ThermoMixer® (500 rpm) at 37° C. for 18 hr. The reaction was cooled to room temperature, and Intermediate Compound 3 (3.5 mM, 114.3 μL) was charged to the 2-mL HPLC vial that was returned to the Eppendorf ThermoMixer® (500 rpm) at 37° C. for 4 hr. The reaction was cooled to room temperature and quenched with EDTA (26.7 mM, 3 mL).

Results: The ligation yielded 8.29 mg of the second RNAi agent (93.97% by UPLC).

### Example 47: Scale-Up of Enzymatic Ligation Using RNA Ligase to Form a RNAi Agent

Synthesis: A first RNAi agent having a sense strand of SEQ ID NO:1 and an antisense strand of SEQ ID NO:2 was synthesized using 0.025 g/L of a third RNA ligase (Codexis) to ligate Intermediate Compounds 1, 2, 3, 4 and 5 (0.4 mM) in the presence of ATP (1.5 mM) and MgCl₂ (3.0 mM). In a 200-mL pressure vial, a reaction buffer (96.52 mL) was prepared by adding Tris-HCl, pH 7.5 (1000 mM, 4.83 mL), MgCl₂ (100 mM, 3.62 mL), DTT (100 mM, 1.25 mL), ATP (10 mM, 18.15 mL), Intermediate Compound 1 (4.5 mM, 10.72 mL), Intermediate Compound 2 (6.7 mM, 7.20 mL), Intermediate Compound 3 (3.6 mM, 13.41 mL), Intermediate Compound 4 (6.0 mM, 8.04 mL) and Intermediate Compound 5 (6.2 mM, 7.78 mL) in nuclease-free water (21.52 mL). An enzyme working solution (0.125 g/L, 24.38 mL) was prepared by diluting RNA ligase (10.16 g/L, 300 μL) in an enzyme storage buffer (24.08 mL). The enzyme working solution (24.13 mL) was added to the reaction buffer (96.52 mL). The reaction mixture was thoroughly mixed by gentle inversion. The 200-mL pressure vessel was equipped with a magnetic stir bar (250 rpm) and an adapter that contained a pressure relief valve, pressure gauge and a thermocouple to monitor internal reaction temperature. The reaction was heated to 37° C. for 21 hr in a water bath with an immersed copper coil that was temperature controlled by ThermoFisher Haake™ Phoenix II chiller/circulator. The reaction was quenched with EDTA (26.7 mM, 360 mL). After 21 hr, most of Intermediate Compounds 1, 2, 3, 16 and 17 were consumed to afford crude first RNAi agent (0.727 g by optical density, 92.72% (IM1).

Results: The ligation yielded 0.587 g of purified second RNAi agent (89.46% (IM2) by UPLC).

### Example 48: Scale-Up of Enzymatic Ligation Using RNA Ligase to Form a RNAi Agent

Synthesis: A first RNAi agent having a sense strand of SEQ ID NO:1 and an antisense strand of SEQ ID NO:2 was synthesized using 1 g/L a fourth RNA ligase (Codexis) to ligate Intermediate Compounds 1, 2, 3, 4 and 5 (0.4 mM) in the presence of ATP (2.0 mM) and MgCl₂ (10.0 mM). A 10 mg/mL enzyme stock solution was prepared by dissolving RNA ligase (125.6 mg) in 50 mM Tris HCl, pH 7.5 (12.56 mL). In a 200-mL pressure vial, a reaction buffer (120.7 mL) was prepared by adding Tris-HCl, pH 7.5 (1000 mM, 6.0 mL), MgCl₂ (100 mM, 12.1 mL), KCl (2000 mM, 6.0 mL), DTT (100 mM, 1.2 mL), ATP (100 mM, 2.4 mL), Intermediate Compound 1 (8.1 mM, 6.0 mL), Intermediate Compound 2 (12.1 mM, 4.0 mL), Intermediate Compound 3 (6.2 mM, 7.8 mL), Intermediate Compound 4 (10.0 mM, 4.8 mL), Intermediate Compound 5 (10.8 mM, 4.5 mL) and RNA ligase (10 mg/mL, 12.1 mL) in nuclease-free water (53.8 mL). The reaction mixture was thoroughly mixed by gentle inversion. The 200-mL pressure vessel was equipped with a magnetic stir bar (250 rpm) and an adapter that contained a pressure relief valve, pressure gauge and a thermocouple to monitor internal reaction temperature. The reaction was heated to 37° C. for 23.5 hr in a water bath with an immersed copper coil that was temperature controlled by ThermoFisher Haake™ Phoenix II chiller/circulator. The reaction was quenched with EDTA (26.7 mM, 54.38 mL). After 21 hr, most of Intermediate Compounds 1, 2, 3, 4 and 5 were consumed to afford crude first RNAi agent (1.01 g by optical density, 86.44% (IM1) by UPLC).

Results: The ligation yielded 0.682 g of purified second RNAi agent (90.39% (IM2) by UPLC).

### Example 49: SPOS of Intermediate Compounds 1-5 at 15 or 22 mmol Scales

Synthesis: Intermediate Compounds 1, 2, 4 and 5, or a pharmaceutically acceptable salt thereof, were synthesized at a 22 mmol-scale by SPOS according to the method parameters outlined in Table 8. Similarly, Intermediate Compound 3, or a pharmaceutically acceptable salt thereof, was synthesized at a 15 mmol-scale by SPOS according to the method parameters outlined in Table 8. All syntheses were performed on an AKTA OligoPilot 400 synthesizer.

TABLE 8
Optimized Method Parameters for SPOS.

| | | Intermediate Compounds | Intermediate |
|---|---|---|---|
| Process Step | Parameter | 1, 2, 4 and 5 | Compound 3 |
| Synthesis | Scale (mmol) | 22.0 | 15.0 |
| Column and | Weight (g) | 62.86 | 42.86 |
| Synthesizer | Support | NittoPhase HL | NittoPhase HL |
| | | Unylinker 350 | Unylinker 350 |
| | Support Loading (μmol/g) | 350 | 350 |
| | Support Specific | 0.109 | 0.109 |
| | Density (g/mL) | | |
| | Column Volume (mL) | 576.98 | 392.5 |
| | Column Diameter (mm) | 100 | 100 |
| | Column Height (cm) | 7.35 | 5.0 |
| | Synthesizer | OP400 | OP400 |
| | UV1 (nm) | 350 | 350 |
| Pre-Cycle | Pre-Synthesis Wash | ACN | ACN |
| Wash | Wash LFlow Rate | 400 | 400 |
| | (cm/hr) | | |
| | Wash Volume (CV) | 4 | 4 |
| Detritylation | Reagent | 3% DCA | 3% DCA |
| | UV Wavelength (nm) | 350 | 350 |
| | Detrit. Charge | 360 | 360 |
| | LFlow Rate (cm/hr) | | |
| | Detrit volume | 1. After 2.0 CV initiate | 1. After 2.0 CV initiate |
| | | UV1 watch at 350 nm. | UV1 watch at 350 nm. |
| | | 2. Start UV1 integration | 2. Start UV1 integration |
| | | at UV trigger >300 mAU. | at UV trigger >300 mAU. |
| | | 3. 4.0 CV after | 3. 4.0 CV after |
| | | integration start, UV1 | integration start, UV1 |
| | | watch at 350 nm. | watch at 350 nm. |
| | | 4. End detritylation delivery | 4. End detritylation delivery |
| | | at UV trigger <300 mAU | at UV trigger <300 mAU |
| | ACN Push LFlow | 360 | 360 |
| | Rate1 (cm/hr) | | |
| | ACN Push volume1 (CV) | 1 | 1 |
| | ACN Wash LFlow | 400 | 400 |
| | Rate2 (cm/hr) | | |
| | ACN Wash | 6 | 6 |
| | volume2 (CV) | | |
| Coupling | Amidite Conc. (M) | 0.2 | 0.2 |
| | Amidite Eq. | 1.5 | 1.5 |
| | Amidite Charge | 165 | 113 |
| | Flow Rate (mL/min) | | |
| | Activator Conc. (M) | 0.6 | 0.6 |
| | Activator Eq. | 6.75 | 6.75 |
| | Activator Charge | 248 | 169 |
| | Flow Rate (mL/min) | | |
| | Amidite/Activator | 1 | 1 |
| | Charge Time (min) | | |
| | Activator and | 412.5 | 281.25 |
| | Amidite Charge | | |
| | Volume (mL) | | |
| | Coupling Push | 20 | 20 |
| | Volume (mL) | | |
| | Coupling Push | Amidite: 165; | Amidite: 113; |
| | Flow Rates (mL/min) | Activator: 248 | Activator: 169 |
| | Recirculation | 136 | 136 |
| | LFlow Rate (cm/hr) | | |
| | Recirculation | 10 (1^{st} coupling twice) | 10 (1^{st} coupling twice; 20 |
| | Time (min) | | for Adem A) |
| | Coupling Wash | 400 | 400 |
| | LFlow Rate (cm/hr) | | |
| | Coupling Wash | 2 | 2 |
| | Volume (CV) | | |
| Thiolation | Reagent | 0.2M Xanthane Hydride | N/A |
| (Only | | in Py | |
| applicable | Thiolation Eq. | 6 | N/A |
| for | | | |
| Intermediate | Thiolation Charge | 110 | N/A |
| Compounds | Flow Rate (mL/min) | | |
| 1, 4, and 5) | Thiolation Contact | 6 | N/A |
| | Time (min) | | |
| | Thiolation Push | 2 | N/A |
| | Volume (CV) | | |
| | Thiolation Push | 110 | N/A |
| | Flow Rate (mL/min) | | |
| | Thiolation Wash | 400 | N/A |
| | LFlow Rate (cm/hr) | | |
| | Thiolation Wash | 2 | N/A |
| | Volume (CV) | | |
| Oxidation | Reagent | 0.05M I₂ in Py/H₂O | 0.05M I₂ in Py/H₂O |
| | Oxidation Eq. | 3 | 3 |
| | Oxidation Charge | 165 | 113 |
| | Flow Rate (mL/min*2) | | |
| | Oxidation Contact | 4 | 4 |
| | Time (min) | | |
| | Oxidation Push | 2 | 2 |
| | Volume (CV) | | |
| | Oxidation Push | 165 | 113 |
| | Flow Rate (mL/min*2) | | |
| | Oxidation Wash | 400 | 400 |
| | LFlow Rate (cm/hr) | | |
| | Oxidation Wash | 2 | 2 |
| | Volume (CV) | | |
| Capping | Reagent | CapA 20% Ac₂O/ACN | CapA 20% Ac₂O/ACN |
| (Only | | CapB 20% NMI/Py/ACN | CapB 20% NMI/Py/ACN |
| applicable | Charge Volume (CV) | 0.5 | 0.5 |
| in 1^{st} cycle | | | |
| when | Charge Flow Rate | CapA: 288; | CapA: 196; |
| loading | (mL/min) | CapB: 288 | CapB: 196 |
| amidite on | Charge Contact | 0.5 (1st capping twice, | 0.5 (1st capping twice, |
| unylinker | Time (min) | after 1^{st} capping, wash | after 1^{st} capping, wash |
| resin) | | the flowpath of CapA | the flowpath of CapA |
| | | and CapB with ACN, | and CapB with ACN, |
| | | other cycles without | other cycles without |
| | | cap step) | cap step) |
| | Capping Push | 2 | 2 |
| | Volume (CV) | | |
| | Capping Push Flow | ACN: 288; | ACN: 196; |
| | Rate (mL/min) | ACN: 288 | ACN: 196 |
| | Capping Wash | 400 | 400 |
| | LFlow Rate (cm/hr) | | |
| | Capping Wash | 2 | 2 |
| | Volume (CV) | | |
| Final | Reagent | 3% DCA | 3% DCA |
| Detritylation | UV Wavelength (nm) | 350 | 350 |
| | Detrit. Charge | 360 | 360 |
| | LFlow Rate (cm/hr) | | |
| | Detrit volume | 1. After 2.0 CV initiate | 1. After 2.0 CV initiate |
| | | UV1 watch at 350 nm. | UV1 watch at 350 nm. |
| | | 2. Start UV1 integration | 2. Start UV1 integration |
| | | at UV trigger >300 mAU. | at UV trigger >300 mAU. |
| | | 3. 4.0 CV after | 3. 4.0 CV after |
| | | integration start, UV1 | integration start, UV1 |
| | | watch at 350 nm. | watch at 350 nm. |
| | | 4. End detritylation delivery | 4. End detritylation delivery |
| | | at UV trigger <300 mAU | at UV trigger <300 mAU |
| | ACN Push LFlow | 360 | 360 |
| | Rate1 (cm/hr) | | |
| | ACN Push | 1 | 1 |
| | volume1 (CV) | | |
| | ACN Wash LFlow | 400 | 400 |
| | Rate2 (cm/hr) | | |
| | ACN Wash | 6 | 6 |
| | volume2 (CV) | | |
| Amine | Reagents | 20% DEA/ACN | 20% DEA/ACN |
| Wash | Charge Volume (CV) | 6 | 6 |
| | Charge Flow Rate | 231 | 157 |
| | (mL/min) | | |
| | Charge Time (min) | 15 | 15 |
| | ACN Push Flow | 231 | 157 |
| | Rate (mL/min) | | |
| | ACN Push | 1 | 1 |
| | Volume (CV) | | |
| | ACN Wash LFlow | 400 | 400 |
| | Rate (cm/hr) | | |
| | ACN Wash | 4 | 4 |
| | Volume (CV) | | |
| Post | Flow Rate | 400 | 400 |
| Synthesis | (cm/hr) | | |
| ACN Wash | Volume (CV) | 4 | 4 |

After synthesis, resin-bound, crude oligonucleotide fragments were dried using nitrogen gas until a consistent mass was achieved. Ammonolysis was performed by adding concentrated NH₄OH (100 ml/mmol) to the crude oligonucleotide fragments on resin. The material was subjected to the cleavage and deprotection conditions shown in Table 9. After 16 hr, spent resin was filtered from the ammonolysis solution to afford the crude, deprotected oligonucleotide fragments in solution.

TABLE 9
C&D Conditions for Intermediate Compounds 1-5.

| | | Intermed. Cmpd. | | | | Intermed. |
|---|---|---|---|---|---|---|
| Parameters | | 1, 2, 4, 5 | | | | Cmpd. 3 |
| C&D | Scale (mmol) | 21.45 | 21.90 | 21.90 | 21.90 | 14.90 |

| | |
|---|---|
| Reagent (%) | ammonium hydroxide (25-28) |
| Temperature (° C.) | 38-42 |
| Time (hr) | 16 |

Results: Synthesis results after cleavage and deprotection (C&D) are displayed in Table 10.

TABLE 10
SPOS Results for Crude Intermediate Compounds 1-5 After C&D.

| | Compound | Intermed. | Intermed. | Intermed. | Intermed. | Intermed. |
|---|---|---|---|---|---|---|
| | ID | Cmpd. 1 | Cmpd. 2 | Cmpd. 3 | Cmpd. 4 | Cmpd. 5 |
| Product | Weight (g) | 4393.40 | 3463.03 | 3439.38 | 4110.00 | 3841.89 |
| | Conc. (OD/g) | 538.88 | 520.74 | 430.34 | 571.98 | 496.16 |
| | Total OD | 2,367,515 | 1,803,338 | 1,480,103 | 2,350,838 | 1,906,192 |
| | Yield (OD/μmol) | 110.37 | 82.34 | 99.33 | 107.34 | 87.04 |
| | Purity (%) | 87.2 | 88.5 | 85.5 | 83.5 | 85.2 |

The ammonolysis solutions were further processed by ultrafiltration and diafiltration (UF/DF) using a Sartorius TFF system equipped with a Sartorius 2 kDa MCWO membrane. A salt exchange was performed using a 0.5 μM NaCl solution. The solutions were desalted until the end conductivity value was achieved. Detailed parameters for the UF/DF process are listed in Table 11. After UF/DF, the products were isolated using lyophilization to afford Intermediate Compounds 1-5 as sodium salts. The final yield and purity for Intermediate Compounds 1-5 are listed in Table 12.

TABLE 11
UF/DF Parameters for Intermediate Compounds 1-5.

| Process | | | | | | |
|---|---|---|---|---|---|---|
| Step | Parameter | Intermed. Cmpds. 1-5 | | | | |
| Cassette | MWCO (kD) | 2 | | | | |
| | Area (m²) | 0.6 | | | | |
| | Number | 3 | | | | |
| | Total Area (m²) | 1.8 | | | | |
| CIP | CIP Solution (M) | 0.5 NaOH | | | | |
| | Time (hr) | 0.5 | | | | |
| | End pH | 6.5-7.5 | | | | |
| | End conduct. (mS/cm) | ≤0.1 | | | | |
| | | Cmpd. ID | | | | |
| | | Intermed. | Intermed. | Intermed. | Intermed. | Intermed. |
| | | Cmpd. 1 | Cmpd. 2 | Cmpd. 3 | Cmpd. 4 | Cmpd. 5 |
| Loading | Total OD | 2,365,899 | 1,801,776 | 1,478,894 | 2,349,122 | 1,904,571 |
| | TMP (bar) | 0.5-0.8 | 0.8-1.2 | 0.8-1.2 | 0.8-1.2 | 0.8 |
| | Feed Flow (L/h) | 15-18 | 20-24 | 20-25 | 20-24 | 18 |
| Salt | Solution (M) | 0.5 NaCl | 0.5 NaCl | 0.5 NaCl | 0.5 NaCl | 0.5 NaCl |
| Exchange | Diavolumes | 10 | 10 | 10 | 10 | 10 |
| | TMP (bar) | 1.0-1.8 | 1.0-1.8 | 1.0-1.4 | 1.0-1.8 | 0.8-1.6 |
| | Feed Flow (L/h) | 24-26 | 25-28 | 25-28 | 25-28 | 24-26 |
| Diafiltration | Permeate Conduct. | ≤0.1 | | | | |
| | Endpoint (mS/cm) | | | | | |
| Combined | Weight (g) | 1765.9 | 1659.21 | 1594.70 | 1599.55 | 1681.63 |
| Solution | Conc. (OD/g) | 1200.25 | 948.52 | 772.03 | 1237.5 | 945.12 |
| | Total ODs | 2,119,630 | 1,573,794 | 1,231,156 | 1,979,443 | 1,589,342 |
| | Total ODs | 89.59 | 87.35 | 83.25 | 84.26 | 83.45 |
| | Recovery (%) | | | | | |
| | Purity (%) | 89.8% | 92.9 | 89.1 | 89.6 | 89.1 |

TABLE 12
Results for Intermediate Compounds 1-5

| Intermed. | Actual Yield | Purity | ID | Assay | Yield |
|---|---|---|---|---|---|
| Cmpd. | (g) | (%) | (Da) | (%) | (%) |
| 1 | 80.07 | 90.2 | 4587.7 | 82.6 | 65.5 |
| 2 | 60.32 | 90.2 | 3363.6 | 84.3 | 68.7 |
| 3 | 62.95 | 86.4 | 5300.4 | 79.2 | 62.7 |
| 4 | 68.86 | 88.4 | 4141.7 | 83.1 | 62.8 |
| 5 | 60.46 | 90.1 | 3378.4 | 82.5 | 67.1 |

### Example 50: 7 Gram Scale-Up of Enzymatic Ligation Using RNA Ligase to Form a RNAi Agent

Synthesis: A first RNAi agent having a sense strand of SEQ ID NO:1 and an antisense strand of SEQ ID NO:2 was synthesized using 0.1 g/L a fifth RNA ligase (Codexis, cell-free extract) to ligate Intermediate Compounds 1, 2, 3, 4 and 5 (0.4 mM) in the presence of ATP (2.0 mM) and MgCl₂ (10.0 mM). In a 2-L pressure vial, a reaction buffer (845.0 mL) was prepared by adding Tris-HCl, pH 7.5 (1 μM, 42.30 mL), MgCl₂ (1 μM, 8.50 mL), KCl (6.299 g), DTT (130.0 mg), ATP disodium salt hydrate (931.0 mg), Intermediate Compound 1 (7.92 mM, 42.67 mL), Intermediate Compound 2 (11.92 mM, 28.35 mL), Intermediate Compound 3 (6.02 mM, 56.15 mL), Intermediate Compound 4 (9.60 mM, 35.21 mL), and Intermediate Compound 5 (11.49 mM, 29.41 mL) in nuclease-free water (602.4 mL). The RNA ligase (84.5 mg) was added to the reaction buffer. The 2-L pressure vessel was equipped with a magnetic stir bar (150 rpm) and an adapter that contained a pressure relief valve, pressure gauge and a thermocouple to monitor internal reaction temperature. The reaction was heated to 37° C. for 25 hr in a water bath with an immersed copper coil that was temperature controlled by ThermoFisher Haake™ Phoenix II chiller/circulator. After 25 hr, the reaction was allowed to cool to room temperature and quenched with EDTA (0.5 μM, 34 mL). Intermediate Compounds 1, 2, 3, 4 and 5 were mostly consumed to afford crude first RNAi agent (83.04% (IM2) by UPLC).

Results: After purification, the ligation yielded 6.62 g (93.82% yield) of potency-corrected the RNAi agent (87.66% (IM2) by UPLC, 92.91% by non-denaturing UPLC method).

### Example 51: 100 Gram Scale-Up of Enzymatic Ligation Using RNA Ligase to Form a RNAi Agent

Synthesis: A first RNAi agent having a sense strand of SEQ ID NO:1 and an antisense strand of SEQ ID NO:2 was synthesized using 0.1 g/L a fifth RNA ligase (Codexis, cell-free extract) to ligate Intermediate Compounds 1, 2, 3, 4 and 5 (0.4 mM) in the presence of ATP (2.0 mM) and MgCl₂ (10.0 mM). In a 22-L, three-neck, jacketed reactor, a reaction buffer (12.066 L) was prepared by adding Tris-HCl, pH 7.5 (1 μM, 603.275 mL), MgCl₂ (1 μM, 120.655 mL), KCl (89.948 g), DTT (1.861 g), ATP disodium salt hydrate (13.300 g), Intermediate Compound 1 (15.37 mM, 314.001 mL), Intermediate Compound 2 (22.97 mM, 210.109 mL), Intermediate Compound 3 (15.26 mM, 316.265 mL), Intermediate Compound 4 (17.37 mM, 277.847 mL) and Intermediate Compound 5 (23.23 mM, 207.757 mL) in nuclease-free water (10.016 L). The RNA ligase (1.207 g) was added to the reaction buffer. The 22-L, three-neck, jacketed reactor was equipped with an overhead stirrer (80 rpm), a baffle, nitrogen gas line, and a thermocouple to monitor internal reaction temperature. A ThermoFisher Haake™ Phoenix II chiller/circulator—containing a 50:50 propylene glyocol/water solution—was plumbed to the fittings on the jacket of the reactor. The reaction was heated to 37° C. for 24.5 hr. After 24.5 hr, the reaction was quenched with EDTA (0.5 μM, 482.62 mL) and allowed to cool to room temperature. Using a peristaltic pump, the crude reaction mixture was filtered through a Millipak 40 Gamma Gold Capsule with a sterile, Durapore membrane (0.22 μm, PVDF). Intermediate Compounds 1, 2, 3, 4 and 5 were mostly consumed to afford crude first RNAi agent (84.1% (IM2) by UPLC).

Results: After purification the ligation yielded 92.20 g (76.16% yield, extrapolated, corrected for purity and water content) of the RNAi agent as a sodium salt (88.87% (IM2) by UPLC, 93.40% by non-denaturing UPLC method).

### Example 52: Standard Conditions for Enzymatic Ligations Using RNA Ligase to Form an RNAi Agent—A Four-Fragment Approach

Synthesis: A first RNAi agent having a sense strand of SEQ ID NO:1 and an antisense strand of SEQ ID NO:2 was synthesized using 0.1 g/L of the fifth RNA ligase (Codexis, cell-free extract) to ligate Intermediate Compounds 1, 32, 4 and 5 (0.4 mM) in the presence of ATP (2 mM) and MgCl₂ (10 mM). A 10 mg/mL enzyme stock solution was prepared by dissolving RNA ligase in nuclease-free water. In a 2-mL HPLC vial, a reaction buffer (1000 μL) was prepared by adding Tris-HCl, pH 7.5 (1000 mM, 50 μL), MgCl₂ (100 mM, 100 μL), KCl (2000 mM, 50 μL), DTT (100 mM, 10 μL), ATP (100 mM, 20 μL), Intermediate Compound 1 (7.92 mM, 50.5 μL), Intermediate Compound 32 (0.92 mM, 435.9 μL), Intermediate Compound 4 (9.60 mM, 41.7 μL), Intermediate Compound 5 (11.49 mM, 34.8 μL) and RNA ligase (10 mg/mL, 10 μL) in nuclease-free water (197.1 μL). The reaction mixture was thoroughly mixed by gently pipetting the solution up and down. The 2-mL HPLC vial was placed in an Eppendorf ThermoMixer® (500 rpm) at 37° C. for 23 hr. The reaction was quenched with EDTA (26.7 mM, 3 mL).

Results: The ligation yielded 8.29 mg (theoretical) of the RNAi agent (88.04% (IM1) by UPLC).

### Example 53: Enzymatic Ligation Using Commercial RNA Ligase to Form an RNAi Agent

Synthesis: A first RNAi agent having a sense strand of SEQ ID NO:1 and an antisense strand of SEQ ID NO:2 was synthesized using 0.025 g/L of the sixth RNA ligase (New England Biolabs, M0239) to ligate Intermediate Compounds 1, 2, 3, 4 and 5 (0.4 mM) in the presence of ATP (2 mM) and MgCl₂ (10 mM). In a 2-mL HPLC vial, a reaction buffer (500 μL) was prepared by adding Tris-HCl, pH 7.5 (1000 mM, 25 μL), MgCl₂ (100 mM, 50 μL), KCl (2000 mM, 25 μL), DTT (100 mM, 5 μL), ATP (100 mM, 10 μL), Intermediate Compound 1 (15.37 mM, 13.0 μL), Intermediate Compound 2 (22.97 mM, 8.7 μL), Intermediate Compound 3 (15.26 mM, 13.1 μL), Intermediate Compound 4 (17.37 mM, 11.5 μL), Intermediate Compound 5 (23.23 mM, 8.6 μL) and RNA ligase (0.25 mg/mL, 50 μL) in nuclease-free water (280.1 μL). The reaction mixture was thoroughly mixed by gently pipetting the solution up and down. The 2-mL HPLC vial was placed in an Eppendorf ThermoMixer® (500 rpm) at 37° C. for 20 hr. The reaction was quenched with EDTA (26.7 mM, 1.5 mL).

Results: The ligation yielded 4.15 mg (theoretical) of the RNAi agent (84.71% (IM2) by UPLC).

### SEQUENCES

The following nucleotide and/or amino acid sequences are referred to in the disclosure and are provided below for reference.

-Artificial Nucleic Acid #1 (36 nt)
SEQ ID NO: 1
mU^{S}-mU-mG-mC-mC-mA-mA-fG-fC-fU-fU-mG-mG-mU-mC-mA-mU-mC-mU-mA-mG-
mC-mA-mG-mC-mC-mG-[ademA-GalNAc]-[ademA-GalNAc]-[ademA-GalNAc]-mG-mG-
mC-mU-mG-mC
-Artificial Nucleic Acid #2 (22 nt)
SEQ ID NO: 2
[MePhosphonate-4O-mU^{S}]-fA^{S}-fG^{S}-fA-fU-mG-fA-mC-mC-fA-mA-mG-mC-fU-mU-mG-
mG-mC-mA-mA^{S}-mG^{S}-mG
-Artificial Nucleic Acid #3 (36 nt)
SEQ ID NO: 3
mU^{S}-mC-mA-mA-mA-mA-mU-fG-fG-fA-fA-mG-mG-mU-mU-mA-mU-mA-mC-mA-mG-
mC-mA-mG-mC-mC-mG-[ademA-GalNAc]-[ademA-GalNAc]-[ademA-GalNAc]-mG-mG-
mC-mU-mG-mC
-Artificial Nucleic Acid #4 (22 nt)
SEQ ID NO: 4
[MePhosphonate-4O-mU^{S}]-fG^{S}-fU^{S}-fA-fU-mA-fA-mC-mC-fU-mU-mC-mC-fA-mU-mU-
mU-mU-mG-mA^{S}-mG^{S}-mG
-Intermediate Compound 1 (14 nt)
SEQ ID NO: 5
mU^{S}-mU-mG-mC-mC-mA-mA-fG-fC-fU-fU-mG-mG-mU
-Intermediate Compound 2 (10 nt)
SEQ ID NO: 6
p-mC-mA-mU-mC-mU-mA-mG-mC-mA-mG
-Intermediate Compound 3 (12 nt)
SEQ ID NO: 7
p-mC-mC-mG-[ademA-GalNAc]-[ademA-GalNAc]-[ademA-GalNAc]-mG-mG-mC-mU-
mG-mC
-Intermediate Compound 4 (12 nt)
SEQ ID NO: 8
p-mA-mG-mC-fU-mU-mG-mG-mC-mA-mA^{S}-mG^{S}-mG
-Intermediate Compound 5 (10 nt)
SEQ ID NO: 9
[MePhosphonate-4O-mU^{S}]-fA^{S}-fG^{S}-fA-fU-mG-fA-mC-mC-fA
-Intermediate Compound 6 (12 nt)
SEQ ID NO: 10
p-mC-mA-mU-mC-mU-mA-mG-mC-mA-mG-mC-mC
-Intermediate Compound 7 (10 nt)
SEQ ID NO: 11
p-mG-[ademA-GalNAc]-[ademA-GalNAc]-[ademA-GalNAc]-mG-mG-mC-mU-mG-mC
-Intermediate Compound 8 (11 nt)
SEQ ID NO: 12
p-mC-mA-mU-mC-mU-mA-mG-mC-mA-mG-mC
-Intermediate Compound 9 (11 nt)
SEQ ID NO: 13
p-mC-mG-[ademA-GalNAc]-[ademA-GalNAc]-[ademA-GalNAc]-mG-mG-mC-mU-mG-
mC
-Intermediate Compound 10 (10 nt)
SEQ ID NO: 14
mU^{S}-mU-mG-mC-mC-mA-mA-fG-fC-fU
-Intermediate Compound 11 (14 nt)
SEQ ID NO: 15
p-fU-mG-mG-mU-mC-mA-mU-mC-mU-mA-mG-mC-mA-mG
-Intermediate Compound 12 (16 nt)
SEQ ID NO: 16
p-fA-mC-mC-fA-mA-mG-mC-fU-mU-mG-mG-mC-mA-mA^{S}-mG^{S}-mG
-Intermediate Compound 13 (6-nt)
SEQ ID NO: 17
[MePhosphonate-4O-mU^{S}]-fA^{S}-fG^{S}-fA-fU-mG
-Intermediate Compound 14 (9 nt)
SEQ ID NO: 18
mU^{S}-mU-mG-mC-mC-mA-mA-fG-fC
-Intermediate Compound 15 (15 nt)
SEQ ID NO: 19
p-fU-fU-mG-mG-mU-mC-mA-mU-mC-mU-mA-mG-mC-mA-mG
-Intermediate Compound 16 (15 nt)
SEQ ID NO: 20
p-mC-mC-fA-mA-mG-mC-fU-mU-mG-mG-mC-mA-mA^{S}-mG^{S}-mG
-Intermediate Compound 17 (7 nt)
SEQ ID NO: 21
[MePhosphonate-4O-mU^{S}]-fA^{S}-fG^{S}-fA-fU-mG-fA
-Intermediate Compound 18 (8 nt)
SEQ ID NO: 22
mU^{S}-mU-mG-mC-mC-mA-mA-fG
-Intermediate Compound 19 (16 nt)
SEQ ID NO: 23
p-fC-fU-fU-mG-mG-mU-mC-mA-mU-mC-mU-mA-mG-mC-mA-mG
-Intermediate Compound 20 (14 nt)
SEQ ID NO: 24
p-mC-fA-mA-mG-mC-fU-mU-mG-mG-mC-mA-mA^{S}-mG^{S}-mG
-Intermediate Compound 21 (8 nt)
SEQ ID NO: 25
[MePhosphonate-4O-mU^{S}]-fA^{S}-fG^{S}-fA-fU-mG-fA-mC
-Intermediate Compound 22 (13 nt)
SEQ ID NO: 26
p-mC-mA-mU-mC-mU-mA-mG-mC-mA-mG-mC-mC-mG
-Intermediate Compound 23 (9 nt)
SEQ ID NO: 27
p-[ademA-GalNAc]-[ademA-GalNAc]-[ademA-GalNAc]-mG-mG-mC-mU-mG-mC
-Intermediate Compound 24 (18 nt)
SEQ ID NO: 28
p-mC-mA-mU-mC-mU-mA-mG-mC-mA-mG-mC-mC-mG-[ademA-GalNAc]-[ademA-
GalNAc]-[ademA-GalNAc]-mG-mG
-Intermediate Compound 25 (4 nt)
SEQ ID NO: 29
mC-mU-mG-mC
-Intermediate Compound 26 (6 nt)
SEQ ID NO: 30
p-mG-mC-mA-mA^{S}-mG^{S}-mG
-Intermediate Compound 27 (6 nt)
SEQ ID NO: 31
p-mA-mG-mC-fU-mU-mG
-Intermediate Compound 28 (14 nt)
SEQ ID NO: 32
mU^{S}-mC-mA-mA-mA-mA-mU-fG-fG-fA-fA-mG-mG-mU
-Intermediate Compound 29 (10 nt)
SEQ ID NO: 33
p-mU-mA-mU-mA-mC-mA-mG-mC-mA-mG
-Intermediate Compound 30 (12 nt)
SEQ ID NO: 34
p-mU-mC-mC-fA-mU-mU-mU-mU-mG-mA^{S}-mG^{S}-mG
-Intermediate Compound 31 (10 nt)
SEQ ID NO: 35
[MePhosphonate-4O-mU^{S}]-fG^{S}-fU^{S}-fA-fU-mA-fA-mC-mC-fU
-Intermediate Compound 32 (22 nt)
SEQ ID NO: 36
p-mC-mA-mU-mC-mU-mA-mG-mC-mA-mG-mC-mC-mG-[ademA-GalNAc]-[ademA-
GalNAc]-[ademA-GalNAc]-mG-mG-mC-mU-mG-mC
-Intermediate Compound 33 (22 nt)
SEQ ID NO: 37
cyclo-(mC-mA-mU-mC-mU-mA-mG-mC-mA-mG-mC-mC-mG-[ademA-GalNAc]-[ademA-
GalNAc]-[ademA-GalNAc]-mG-mG-mC-mU-mG-mC)

1. An oligonucleotide comprising a nucleotide sequence selected from the group consisting of SEQ ID NOS:5 to 37, or a pharmaceutically acceptable salt thereof.

2. The oligonucleotide of claim 1, wherein the nucleotide sequence is SEQ ID NO:5.

3. The oligonucleotide of claim 1, wherein the nucleotide sequence is SEQ ID NO:6.

4. The oligonucleotide of claim 1, wherein the nucleotide sequence is SEQ ID NO:7.

5. The oligonucleotide of claim 1, wherein the nucleotide sequence is SEQ ID NO:8.

6. The oligonucleotide of claim 1, wherein the nucleotide sequence is SEQ ID NO:9.

7. The oligonucleotide of claim 1, wherein the nucleotide sequence is SEQ ID NO:10.

8. The oligonucleotide of claim 1, wherein the nucleotide sequence is SEQ ID NO:11.

9. The oligonucleotide of claim 1, wherein the nucleotide sequence is SEQ ID NO:12.

10. The oligonucleotide of claim 1, wherein the nucleotide sequence is SEQ ID NO:13.

11. The oligonucleotide of claim 1, wherein the nucleotide sequence is SEQ ID NO:14.

12. The oligonucleotide of claim 1, wherein the nucleotide sequence is SEQ ID NO:15.

13. The oligonucleotide of claim 1, wherein the nucleotide sequence is SEQ ID NO:16.

14. The oligonucleotide of claim 1, wherein the nucleotide sequence is SEQ ID NO:17.

15. The oligonucleotide of claim 1, wherein the nucleotide sequence is SEQ ID NO:18.

16. The oligonucleotide of claim 1, wherein the nucleotide sequence is SEQ ID NO:19.

17. The oligonucleotide of claim 1, wherein the nucleotide sequence is SEQ ID NO:20.

18. The oligonucleotide of claim 1, wherein the nucleotide sequence is SEQ ID NO:21.

19. The oligonucleotide of claim 1, wherein the nucleotide sequence is SEQ ID NO:22.

20. The oligonucleotide of claim 1, wherein the nucleotide sequence is SEQ ID NO:23.

21. The oligonucleotide of claim 1, wherein the nucleotide sequence is SEQ ID NO:24.

22. The oligonucleotide of claim 1, wherein the nucleotide sequence is SEQ ID NO:25.

23. The oligonucleotide of claim 1, wherein the nucleotide sequence is SEQ ID NO:26.

24. The oligonucleotide of claim 1, wherein the nucleotide sequence is SEQ ID NO:27.

25. The oligonucleotide of claim 1, wherein the nucleotide sequence is SEQ ID NO:28.

26. The oligonucleotide of claim 1, wherein the nucleotide sequence is SEQ ID NO:29.

27. The oligonucleotide of claim 1, wherein the nucleotide sequence is SEQ ID NO:30.

28. The oligonucleotide of claim 1, wherein the nucleotide sequence is SEQ ID NO:31.

29. The oligonucleotide of claim 1, wherein the nucleotide sequence is SEQ ID NO:32.

30. The oligonucleotide of claim 1, wherein the nucleotide sequence is SEQ ID NO:33.

31. The oligonucleotide of claim 1, wherein the nucleotide sequence is SEQ ID NO:34.

32. The oligonucleotide of claim 1, wherein the nucleotide sequence is SEQ ID NO:35.

33. The oligonucleotide of claim 1, wherein the nucleotide sequence is SEQ ID NO:36.

34. The oligonucleotide of claim 1, wherein the nucleotide sequence is SEQ ID NO:37.

35. A method of making a nucleic acid of SEQ ID NO:1, the method comprising the step of:
ligating three oligonucleotide fragments selected from the group consisting of:
(a) SEQ ID NOS:5, 6 and 7,
(b) SEQ ID NOS:5, 10 and 11,
(c) SEQ ID NOS:5, 12 and 13,
(d) SEQ ID NOS:7, 14 and 15,
(e) SEQ ID NOS:7, 18 and 19,
(f) SEQ ID NOS:7, 22 and 23, and
(g) SEQ ID NOS:5, 26 and 27.

36. The method of claim 35, wherein the three oligonucleotide fragments are (a) SEQ ID NOS:5, 6 and 7, and wherein the fragments are ligated from 5′ end to 3′ end as SEQ ID NO:5 to SEQ ID NO:6 to SEQ ID NO:7.

37. The method of claim 35, wherein the three oligonucleotide fragments are (b) SEQ ID NOS:5, 10 and 11, and wherein the fragments are ligated from 5′ end to 3′ end as SEQ ID NO:5 to SEQ ID NO:10 to SEQ ID NO:11.

38. The method of claim 35, wherein the three oligonucleotide fragments are (c) SEQ ID NOS:5, 12 and 13, and wherein the fragments are ligated from 5′ end to 3′ end as SEQ ID NO:5 to SEQ ID NO:12 to SEQ ID NO:13.

39. The method of claim 35, wherein the three oligonucleotide fragments are (d) SEQ ID NOS:7, 14 and 15, and wherein the fragments are ligated from 5′ end to 3′ end as SEQ ID NO:14 to SEQ ID NO:15 to SEQ ID NO:7.

40. The method of claim 35, wherein the three oligonucleotide fragments are (e) SEQ ID NOS:7, 18 and 19, and wherein the fragments are ligated from 5′ end to 3′ end as SEQ ID NO:18 to SEQ ID NO:19 to SEQ ID NO:7.

41. The method of claim 35, wherein the three oligonucleotide fragments are (f) SEQ ID NOS: 7, 22 and 23, and wherein the fragments are ligated from 5′ end to 3′ end as SEQ ID NO:22 to SEQ ID NO:23 to SEQ ID NO:7.

42. The method of claim 35, wherein the three oligonucleotide fragments are (g) SEQ ID NOS: 5, 26 and 27, and wherein the fragments are ligated from 5′ end to 3′ end as SEQ ID NO:5 to SEQ ID NO:26 to SEQ ID NO:27.

43. A method of making a nucleic acid of SEQ ID NO:2, the method comprising the step of:
ligating two oligonucleotide fragments selected from the group consisting of:
(a′) SEQ ID NOS:8 and 9;
(b′) SEQ ID NOS:16 and 17;
(c′) SEQ ID NOS:20 and 21; and
(d′) SEQ ID NOS:24 and 25.

44. The method of claim 43, wherein the two oligonucleotide fragments are (a′) SEQ ID NOS:8 and 9, and wherein the fragments are ligated from 5′ end to 3′ end as SEQ ID NO:9 to SEQ ID NO:8.

45. The method of claim 43, wherein the two oligonucleotide fragments are (b′) SEQ ID NOS:16 and 17, and wherein the fragments are ligated from 5′ end to 3′ end as SEQ ID NO:17 to SEQ ID NO:16.

46. The method of claim 43, wherein the two oligonucleotide fragments are (c′) SEQ ID NOS:20 and 21, and wherein the fragments are ligated from 5′ end to 3′ end as SEQ ID NO:21 to SEQ ID NO:20.

47. The method of claim 43, wherein the two oligonucleotide fragments are (d′) SEQ ID NOS:24 and 25, and wherein the fragments are ligated from 5′ end to 3′ end as SEQ ID NO:25 to SEQ ID NO:24.

48. A method of making a nucleic acid of SEQ ID NO:3, the method comprising the step of:
ligating three oligonucleotide fragments of (a) SEQ ID NOS:7, 32 and 33, wherein the fragments are ligated from 5′ end to 3′ end as SEQ ID NO:32 to SEQ ID NO:33 to SEQ ID NO:7.

49. A method of making a nucleic acid of SEQ ID NO:4, the method comprising the step of:
ligating two oligonucleotide fragments of (a′) SEQ ID NOS:34 and 35, wherein the fragments are ligated from 5′ end to 3′ end as SEQ ID NO:35 to SEQ ID NO:34.

50. A method of making a RNAi agent having a sense strand of SEQ ID NO:1 and an antisense strand of SEQ ID NO:2, the method comprising the steps of:
forming the sense strand of SEQ ID NO:1 by ligating three oligonucleotide fragments selected from the group consisting of:
(a) SEQ ID NOS:5, 6 and 7, wherein the fragments are ligated from 5′ end to 3′ end as SEQ ID NO:5 to SEQ ID NO:6 to SEQ ID NO:7,
(b) SEQ ID NOS:5, 10 and 11, wherein the fragments are ligated from 5′ end to 3′ end as SEQ ID NO:5 to SEQ ID NO:10 to SEQ ID NO:11,
(c) SEQ ID NOS:5, 12 and 13, wherein the fragments are ligated from 5′ end to 3′ end as SEQ ID NO:5 to SEQ ID NO:12 to SEQ ID NO:13,
(d) SEQ ID NOS:7, 14 and 15, wherein the fragments are ligated from 5′ end to 3′ end as SEQ ID NO:14 to SEQ ID NO:15 to SEQ ID NO:7,
(e) SEQ ID NOS:7, 18 and 19, wherein the fragments are ligated from 5′ end to 3′ end as SEQ ID NO:18 to SEQ ID NO:19 to SEQ ID NO:7,
(f) SEQ ID NOS: 7, 22 and 23, wherein the fragments are ligated from 5′ end to 3′ end as SEQ ID NO:22 to SEQ ID NO:23 to SEQ ID NO:7, and
(g) SEQ ID NOS: 5, 26 and 27, wherein the fragments are ligated from 5′ end to 3′ end as SEQ ID NO:5 to SEQ ID NO:26 to SEQ ID NO:27;
forming the antisense strand of SEQ ID NO:2 by ligating two oligonucleotide fragments selected from the group consisting of:
(a′) SEQ ID NO:8 and 9, wherein the fragments are ligated from 5′ end to 3′ end as SEQ ID NO:9 to SEQ ID NO:8
(b′) SEQ ID NOS:16 and 17, wherein the fragments are ligated from 5′ end to 3′ end as SEQ ID NO:17 to SEQ ID NO:16,
(c′) SEQ ID NOS:20 and 21, wherein the fragments are ligated from 5′ end to 3′ end as SEQ ID NO:21 to SEQ ID NO:20, and
(d′) SEQ ID NOS:24 and 25, wherein the fragments are ligated from 5′ end to 3′ end as SEQ ID NO:25 to SEQ ID NO:24; and
forming the RNAi agent by annealing complementary nucleotides of the sense strand of SEQ ID NO:1 and the antisense strand of SEQ ID NO:2.

51. A method of making a RNAi agent having a sense strand of SEQ ID NO:3 and an antisense strand of SEQ ID NO:4, the method comprising the steps of:
forming the sense strand of SEQ ID NO:3 by ligating three oligonucleotide fragments of (a) SEQ ID NOS:7, 32 and 33, wherein the fragments are ligated from 5′ end to 3′ end as SEQ ID NO:32 to SEQ ID NO:33 to SEQ ID NO:7;
forming the antisense strand of SEQ ID NO:4 by ligating two oligonucleotides of (a′) SEQ ID NOS:34 and 35, wherein the fragments are ligated from 5′ end to 3′ end as SEQ ID NO:35 to SEQ ID NO:34; and
forming the RNAi agent by annealing complementary nucleotides of the sense strand of SEQ ID NO:3 and the antisense strand of SEQ ID NO:4.

52. The method of claim 35, wherein the ligating step is mediated by an enzyme.

53. The method of claim 52, wherein the enzyme is a deoxyribonucleic acid (DNA) ligase or a ribonucleic acid (RNA) ligase.

54. The method of claim 52, wherein the enzyme is a RNA ligase and is selected from group consisting of RNA Ligase 1 and RNA Ligase 2.
